(19) United States
(12) Patent Application Publication
(10) Pub. No.: US 2005/0186264 A1
(43) Pub. Date: Aug. 25, 2005

(54) Targeting drug/gene carriers to irradiated tissue

(76) Inventors: Mohammad F. Kiani, Wayne, PA (US); Douglas J. Goetz, Athens, OH (US)

Correspondence Address: Benjamin Aaron Adler;ADLER & ASSOCIATES, 8011 Candle Lane, Houston, TX, 77071, US

(21) Appl. No.: 10/993,825

(22) Filed: Nov. 19, 2004

Related U.S. Application Data
(63) Continuation-in-part of application No. 09/975,899, filed on Oct. 12, 2001.
(60) Provisional application No. 60/239,666, filed on Oct. 12, 2000.

(51) Int. Cl. A61K 39/395; A61K 9/127; C07K 16/28

(52) U.S. Cl. 424/450; 424/144.1; 530/388.22; 530/391.1

(57) ABSTRACT

The present invention provides targeted delivery systems to deliver pharmaceuticals to irradiated tissue comprising a biomolecule carrier, a targeting moiety to cellular adhesion molecules and a pharmaceutical. The present invention also provides methods of selectively targeting endothelial tissue for delivery of a pharmaceutical thereto and of treating a pathophysiological state in an individual using the targeted delivery systems disclosed herein. Further provided is a method of optimizing an immunoliposome for specific targeting of a pharmaceutical encapsulated therein to irradiated tissue by selecting a liposome that has a greater rate of adhesion to the irradiated tissue than a rate of uptake by the reticuloendothelial system.

### CROSS-REFERENCE TO RELATED APPLICATION

This application is a continuation-in-part of U.S. Ser. No. 09/975,899, filed Oct. 12, 2001, which claims benefit of provisional U.S. Ser. No. 60/239,666, filed Oct. 12, 2000, now abandoned.

### FEDERAL FUNDING LEGEND

This invention was produced in part using funds obtained through a grant from the National Institutes of Health. Consequently, the federal government has certain rights in this invention.

### BACKGROUND OF THE INVENTION

1. Field of the Invention

The present invention relates generally to the fields of radiation and clinical oncology, radiotherapy, radioimmunobiology and nuclear medicine. More specifically, the present invention relates to a technique of targeting drug or gene carriers to select tissue via the up-regulation of adhesion molecules expressed on endothelial cells in response to exposure to radiation.

2. Description of the Related Art

Ionizing radiation (IR) is used widely to treat many conditions including cancer, arteriovenous malformations (AVM), macular degeneration, and intimal hyperplasia. Ionizing radiation therapy causes vascular lesions and damage in normal tissues. The microvasculature is quite sensitive to radiation (20) and is an important radiation dose-limiting factor in clinical applications. In almost all cases of therapeutic approach, the goal is to limit the exposure of normal tissue to the ionizing radiation while maximizing exposure to the diseased tissue. Indeed, improvement of techniques such as dose fractionation and conformal therapy (68), discovery of radioprotective drugs (78), and development of experimental methods of radiation therapy such as Microbeam Radiation Therapy (70) for reducing normal tissue toxicity of radiotherapy are currently active areas of research. In most cases using modern clinical radiotherapeutic techniques, radiation damage can be limited to a core of diseased tissue and to the immediate normal tissue surrounding it.

Ionizing radiation damage to the microcirculation is manifested in many forms, including increased capillary permeability and up-regulation of inflammatory processes. An increase in permeability is an early and universal response of the microvasculature to ionizing radiation (19,46,50-51,75). For example, permeability in the blood-brain-barrier increases in response to irradiation (22,62). Although this can lead to extravasation of blood proteins, which may exacerbate tissue injury, the increased permeability can enhance delivery of chemotheraputic drugs across the blood-brain-barrier (61-62,64). Therefore, targeted drug delivery to irradiated tissue not only provides a means to deliver the drug selectively, but also delivers the drug to a site of increased vascular permeability.

It has been known for over 15 years that exposure of normal and diseased tissue to irradiation causes an increase in leukocyte infiltration of the tissues (1,8,44,53,65,76). A key component of this process is the adhesion of leukocytes to the microvascular endothelium. A variety of studies focused on elucidating a detailed understanding of leukocyte adhesion in general, i.e. in response to stimuli other than radiation, have revealed that the movement of leukocytes from within the vasculature to the extravascular space involves a well orchestrated set of adhesion events (10,43,49,72). This adhesion cascade is mediated in part by adhesive bonds forming between glycoproteins, i.e., ligands, present on the leukocytes and cognate glycoproteins, i.e., receptors, present on the endothelium.

A key paradigm in this adhesion cascade is that certain endothelial cell adhesion molecules are inducible. That is, they are expressed at a low level, if at all, on endothelium within normal tissue, but are up-regulated dramatically in response to appropriate biochemical stimuli, e.g. cytokines such as IL-1β(10). Thus, in response to various cytokines, the endothelium becomes activated and increases its expression of receptors that bind ligands on the leukocytes. These receptors include E-selectin (CD62E), P-selectin (CD62P), VCAM-1 (CD54) and ICAM-1 (CD106).

Leukocytes attach to the endothelium, for the most part via the selectins, and begin to translate or roll along the vessel wall at a velocity which is significantly lower than leukocytes in the free stream (72). As the leukocytes roll, they become activated in response to chemokines (18,72). The activation involves a number of changes to the leukocytes including an alteration in the density of the integrins on the leukocyte surface as well as an increase in the “stickiness”, i.e., a conformational change, of the integrins for their cognate endothelial cell adhesion molecules, e.g. ICAM-1, (15,56). The leukocytes firmly adhere to the endothelium via the integrins and proceed to migrate between adjacent endothelial cells into the extravascular space in part via PECAM-1 (CD31).

As noted above, a key component of leukocyte emigration is endothelial cell activation which alters the adhesion molecule profile on the lumenal surface of the endothelium. Recognition of these drastically different endothelial surfaces has lead to the concept of endothelial cell adhesion molecule mediated targeted drug delivery (3-4,6-7,16,71). In this therapeutic approach, a drug would be incorporated into a carrier, e.g. a liposome (3-4,7,71) or a biodegradable particle (16,28). The carrier would have a ligand for an endothelial cell adhesion molecule, e.g. E-selectin, that is selectively expressed on the target endothelial segment. Ideally the carrier would bind to the target endothelial segment, e.g. endothelium within a site of inflammation, via the selectively expressed receptor and not bind to non-target endothelium.

It is reasonable to anticipate that some of the molecular mechanisms involved in inflammatory processes initiated by insults other than radiation will also be operative in radiation induced inflammation. Recent literature suggests that this is, at least in part, true. In vitro studies aimed at characterizing the response of endothelial cells to irradiation have consistently shown ICAM-1 up-regulation on endothelial cells derived from large vessels (21,32,73) and vessels of the microvasculature (2,41). In vivo studies have found up-regulation of ICAM-1 (12,35-36,42,47,53,58) and have ascribed increased leukocyte adhesion to the endothelium to an up-regulation of ICAM-1 (53,59). Indeed, radiation induced inflammatory response is attenuated significantly in mice deficient in ICAM-1 relative to wild type mice (35). In a recent clinical study (39) a significant increase in ICAM-1 expression in head and neck cancer patients treated with fractionated radiotherapy of 30-60 Gy in 2 Gy daily fractions has been reported.

At present the expression of E-selectin in response to radiation remains controversial. The expression of E-selectin has been studied in vitro using endothelial cells derived from large veins, such as human umbilical vein endothelial cells (HUVEC). One group reported significant up-regulation of E-selectin on HUVEC (31-33). In addition, it was found that the irradiated human umbilical vein endothelial cells supported E-selectin dependent adhesion of the leukocytic HL-60 cell line in semi-static adhesion assays (33). In contrast, others have found that E-selectin is not up-regulated on human umbilical vein endothelial cells in response to radiation (60,21). It also has been found that irradiated human umbilical vein endothelial cells do not support the adhesion of HL-60 cells under in vitro flow conditions designed to mimic conditions present in vivo.

Specifically, no adhesion of HL-60 cells was observed at shear stresses between 0.5-2.0 dynes/cm² on post-IR human umbilical vein endothelial cells. Note that the lowest physiologically relevant in vitro shear stress is thought to be 0.5 dyne/cm² (26). In contrast to the data on endothelial cells derived from large vein, i.e. HUVEC, a modest up-regulation of E-selectin on human dermal microvascular endothelial cells (HDMEC) was observed which is in agreement with Heckman et al. (41). Consistent with this finding, in vitro flow adhesion assays revealed that post-IR HDMEC did support a small increase in HL-60 cell adhesion at relatively low, e.g. <1.5 dynes/cm², fluid shear.

In vivo, it has been observed that there is an increase in the number of leukocytes that roll along the vessel wall in response to radiation (1,53,59). Consistent with this finding, E-selectin has been found within the microvasculature of the lung in response to radiation (36). A significant increase in E-selectin expression in head and neck cancer patients treated with fractionated radiotherapy of 30-60 Gy in 2 Gy daily fractions has been reported (39).

A few studies have probed for the presence of VCAM-1 in response to radiation in vitro. VCAM-1 was observed to be up-regulated in irradiated skin microvascular endothelium (41), but not irradiated human umbilical vein endothelial cells (21,32). VCAM-1 was not up-regulated in head and neck cancer patients undergoing radiotherapy (39).

The expression of P-selectin post-IR has also been probed. One report found that P-selectin is localized to the vascular lumen of several irradiated tumors in vivo and increases in a time dependent manner until 24 hours post-IR (34). P-selectin is also reportedly translocated to the cell membrane in human umbilical vein endothelial cells within 30 minutes post-IR in vitro and in vivo. It is accumulated in the lumen of irradiated blood vessels in the lung and intestine, but not in the brain or kidney (30,34,37).

Surface protein and mRNA levels of PECAM-1 (CD31), which is involved in the adhesion and transendothelial migration of leukocytes, has been shown to be up-regulated after irradiation in both human umbilical vein endothelial cells and tissue specimens from radiotherapy patients (63), but not in HDMEC (41). The up-regulation of PECAM-1 was accompanied with increased transendothelial migration of leukocytes post-IR and this increased migration was inhibited with a mAb to PECAM-1 (63).

Although the issue of which endothelial cell adhesion molecules are expressed in response to radiation remains controversial, it is abundantly clear that the endothelial cell adhesion molecule profile is significantly altered in response to radiation. There is very convincing evidence that ICAM-1 and PECAM-1 are up-regulated. Although less clear, there is a modest amount of data suggesting that E-selectin is up-regulated as well. Even more noteworthy is that both ICAM-1 and E-selectin were significantly up-regulated in oral mucosa of head and neck cancer patients treated with radiotherapy of 30-60 Gy in 2 Gy daily fractions (39). The radiation-induced up-regulation of endothelial cell adhesion molecules provides the opportunity to target drugs to select tissue via a combination of radiation and ligand-receptor drug targeting technology.

To clarify how the radiation therapy-targeted drug delivery scheme might work, consider the treatment of cancer as an example. Cancer patients are often treated with radiotherapy, chemotherapy or a combination of both. In an effort to limit side effects, the radiotherapy is designed to maximize radiation exposure to the cancerous tissue while minimizing exposure to normal tissue. Similarly, it would be ideal for a chemotherapeutic agent or a gene to be delivered only to the cancerous tissue and not to healthy tissue. Indeed, achieving this goal is the focus of a variety of drug delivery research.

In the combination radiation/targeting therapeutic model, a ligand-bearing drug carrier would be administered subsequent to, or in conjunction with, the radiotherapy. A variety of materials could be used for the drug carrier including liposomes or carriers made from biodegradable polymers. The drug carrier would contain a therapeutic agent, e.g. an organic compound or a nucleic acid, and, on its outer surface, a recognition molecule or ligand for a cognate molecule or receptor that is expressed selectively, due to exposure to the radiation, on the lumenal surface of the endothelium within the irradiated tissue. Ideally, these carriers would bind predominately within the vasculature of the irradiated tissue, such as cancerous tissue, and not bind to the vasculature of normal tissue. In this manner, the radiation-induced up-regulation of a endothelial cell adhesion molecule(s) within the diseased tissue is used as a target to deliver therapeutic agents, e.g., drugs, genes, selectively to the site of disease.

The prior art is deficient in the ability to target drug or gene carriers to select tissue via the up-regulation of adhesion molecules expressed on endothelial cells in response to exposure to radiation. The present invention fulfills this long-standing need and desire in the art.

### SUMMARY OF THE INVENTION

The present invention is directed to a targeted delivery system. The targeted delivery system comprises a biomolecular carrier, a targeting moiety specific for cellular adhesion molecules expressed on endothelial cells conjugated to the biomolecular carrier and a pharmaceutical contained by the biomolecular carrier.

The present invention is directed to a related targeted delivery system. The targeted delivery system comprises a liposome, a cyclic Arg-Gly-Asp-D-Phe-Cys sequence conjugated to the liposome and a pharmaceutical encapsulated by said liposome.

The present also is directed to a method of selectively targeting endothelial tissue for delivery of a pharmaceutical to an individual. The method comprises irradiating a target tissue or organ in the individual to express cellular adhesion molecules on a luminal surface of endothelial tissue comprising the irradiated target tissue or organ, administering the targeted delivery system comprising the pharmaceutical and described herein is administered to the individual whereupon the targeting moiety comprising the targeted delivery system selectively binds the to the cellular adhesion molecule thereby delivering the pharmaceutical to the individual.

The present invention is directed to a related method of treating a pathophysiological state in an individual in need of such treatment. The method comprises irradiating a target tissue or organ characterized by the pathophysiological state in said individual and administering to the individual the targeted delivery system described herein. A pharmaceutical comprising the targeted delivery system is delivered to the irradiated target tissue or organ thereby treating the pathophysiological state in the individual.

The present invention is directed further to a method for optimizing an immunoliposome for specific targeting of a pharmaceutical to irradiated tissue. The method comprises selecting at least one lipid or liposomal component and a targeting moiety such that the combination thereof forms a targeted liposome having a rate of adhesion (Kad) to the irradiated tissue greater than a rate of uptake (Kres) by the reticuloendothelial system. Thereby a majority of the targeted liposomes specifically adhere to the irradiated tissue to deliver the pharmaceutical thereto.

Other and further aspects, features, and advantages of the present invention will be apparent from the following description of the embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate embodiments of the invention and therefore are not to be considered limiting in their scope.

FIG. 1 shows a schematic of the proposed targeted drug delivery scheme.

FIGS. 2A-2B show the up-regulation of E-selectin (FIG. 2A) and of ICAM-1 (FIG. 2B) on irradiated (10Gy) endothelium.

FIGS. 3A-3C show typical pictures of Rhodamine-6G labeled leukocytes in control (FIG. 3A) and 10Gy irradiated (FIG. 3B) cerebral microvasculature and show the up-regulation of leukocyte adhesion in 10Gy irradiated cerebral microvasculature (FIG. 3C).

FIG. 4 shows adhesion of antibody bearing nanospheres to CHO-E.

FIG. 5 shows the selective adhesion of biodegradeable microspheres to activated human umbilical vein endothelial cells.

FIG. 6 shows the adhesion of anti-ICAM-1 microspheres to irradiated human umbilical vein endothelial cells under shear flow (1.5 dynes/cm2).

FIGS. 7A-7D show the adhesion of anti-ICAM-1 (FIG. 7A) and IgG (FIG. 7B) microspheres to irradiated (10Gy) cerebral microvasculature, the adhesion of anti-ICAM-1 microspheres to control cerebral microvasculature before irradiation (FIG. 7C) and the adhesion of anti-ICAM-1 and IgG microspheres to control and irradiated (10Gy) cerebral microvasculature (FIG. 7D).

FIG. 8 shows the adhesion of anti-ICAM-1 and IgG microspheres to the unirradiated microvasculature of the cremaster muscle and to irradiated microvasculature in the brain.

FIGS. 9A-9B show the adhesion of anti-E-selectin (FIG. 9A) and very few IgG (FIG. 9B) microspheres to irradiated (10Gy) cerebral microvasculature.

FIG. 10 shows the adhesion of anti-P-selectin and IgG coated microspheres to 10 Gy irradiated cerebral microvasculature.

FIG. 11 shows the adhesion of PEGylated microspheres to 10 Gy irradiated tumors and non-irradiated tumor and liver in the same animal.

FIGS. 12A-12B show the results of treatment of medium size (FIG. 12A) and larger size (FIG. 12B) B16-F10 melanoma tumors with liposomes (Lipo), Lipo+5Gy radiation (IR).

FIGS. 13A-13D show the results of treatment of spontaneous tumors with immunoiposomes (IL) and IL+5Gy radiation (IR).

FIGS. 14A-14B show low magnification (4×) images of fluorescent (DiOC7) labeling of perfused vessel in untreated (FIG. 14A) and treated (FIG. 14B) B16-F10 tumors.

FIGS. 15A-15B show the percentage of immunoliposomes that are taken up by the RES (FIG. 15A) or bind to the irradiated endothelium (FIG. 15B) as a function of dimensionless time.

### DETAILED DESCRIPTION OF THE INVENTION

In one embodiment of the present invention there is provided a targeted delivery system, comprising a biomolecular carrier; a targeting moiety specific for cellular adhesion molecules expressed on endothelial cells conjugated to the biomolecular carrier; and a pharmaceutical contained by the biomolecular carrier. Further to this embodiment the targeted delivery system may comprise a functionalized poly(ethylene)glycol copolymer on the biomolecular carrier linked to the targeting moiety. An example of such a functional group is maleimide.

In all aspects of this embodiment the biomolecular carrier may be a biodegradable particle, a liposome, a microbubble, a polymersome, or a synthetic secretory granule. Also, in these aspects the cellular adhesion molecule is an integrin, ICAM-1, E-selectin, P-selectin, VCAM-1, or PECAM-1. An example of an integrin is an integrin β3 chain. Furthermore, the targeting moiety may be an antibody or fragment thereof or ligands that bind to said cellular adhesion molecule. An example of a ligand is one that binds to the integrin β3 chain. A representative example of a ligand binding to a β3 chain is a cyclic Arg-Gly-Asp-D-Phe-Cys peptide.

Additionally, in all aspects the pharmaceutical may be an anti-neoplastic compound, an anti-angiogenic compound or a therapeutic genetic macromolecule. An example of an anti-angiogenic compound is combretastatin and 5,6-dimethylxanthenone-4-acetic acid (DMXAA) or a prodrug thereof. An example of a therapeutic genetic macromolecule is a gene.

In a particular aspect of this embodiment the targeted delivery system, the biomolecular carrier may comprise a liposome, the targeting moiety may comprise a cyclic Arg-Gly-Asp-D-Phe-Cys peptide and the pharmaceutical may comprise an anti-angiogenic compound. An example of an anti-angiogenic compound is combretastatin and 5,6-dimethylxanthenone-4-acetic acid (DMXAA) or a prodrug thereof.

In a related embodiment there is provided a targeted delivery system comprising a liposome, a cyclic Arg-Gly-Asp-D-Phe-Cys peptide conjugated to the liposome and a pharmaceutical encapsulated by the liposome. The liposome may comprise a phosphatidyl choline, cholesterol and a diacylphosphatidylethanoloamine-poly(ethylene)glycol conjugate. An example of a phosphatidyl choline is hydrogenated soy phosphatidylcholine. An example of a diacylphosphatidylethanoloamine-poly(ethylene)glycol conjugate is distearoylphosphoethanolamine-PEG(2000). The pharmaceutical may be an anti-neoplastic compound or an anti-angiogenic compound as described supra.

In another embodiment of the present invention there is provided a method of selectively targeting endothelial tissue for delivery of a pharmaceutical to an individual, comprising irradiating a target tissue or organ in the individual; expressing cellular adhesion molecules on a luminal surface of endothelial tissue comprising the irradiated target tissue or organ; administering the targeted delivery system described supra comprising the pharmaceutical to the individual; and selectively binding the targeting moiety comprising said targeted delivery system to said cellular adhesion molecule thereby delivering the pharmaceutical to said individual. Further to this embodiment the method comprises treating a pathophysiological state of the target tissue or organ with the pharmaceutical.

In all aspects of this embodiment the pathophysiological state may be a cancer, arteriovenous malformations, macular degeneration or restenosis. Also, all aspects of the targeted delivery system are described supra. Particularly, the biomolecular carriers, the targeting moieties, the pharmaceuticals, and the cellular adhesion molecules are as described.

In a related embodiment there is provided a method of treating a pathophysiological state in an individual in need of such treatment, comprising irradiating a target tissue or organ characterized by the pathophysiological state in the individual; administering the targeted delivery system described supra to said individual; and delivering the pharmaceutical comprising said targeted delivery system to said irradiated target tissue or organ thereby treating the pathophysiological state in the individual. The pathophysiological states and the components of the targeting system are as described supra.

In yet another embodiment of the present invention there is provided a method of optimizing an immunoliposome for specific targeting of a pharmaceutical encapsulated therein to irradiated tissue, comprising selecting at least one lipid or liposomal component and a targeting moiety such that the combination thereof forms a targeted liposome having a rate of adhesion (K_{ad}) to the irradiated tissue greater than a rate of uptake (Kᵣₑₛ) by the reticuloendothelial system wherein a majority of the targeted liposomes specifically adhere to the irradiated tissue thereby delivering the pharmaceutical thereto. In all aspects of this embodiment a ratio (τ) of K_{ad}/Kᵣₑₛ is greater than 1. In a particular aspect the ratio (τ) is greater than 5.

In a related embodiment there is provided a targeted liposome produced by the optimizing method described supra.

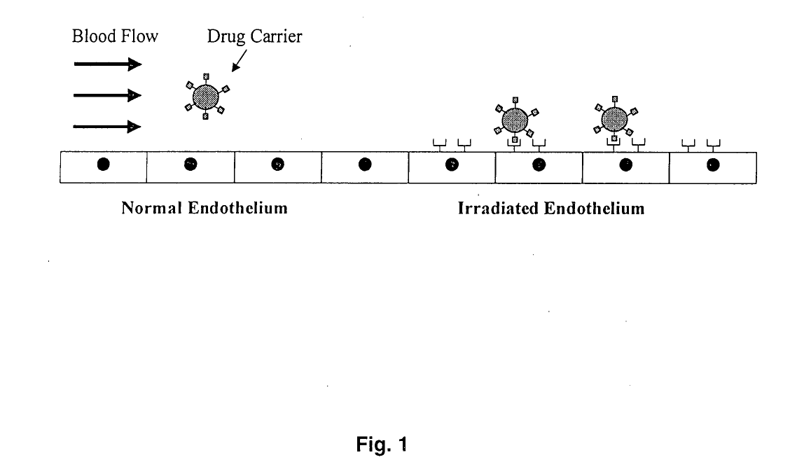

Provided herein is a targeted delivery system and methods of drug delivery for selectively targeting drug or gene biomolecular carriers to tissue that has been irradiated for therapeutic purposes (FIG. 1). The present invention further provides a mathematical model useful to optimize an immunoliposome for specific targeted delivery of a pharmaceutical. Also provided are the optimized liposomal compositions predicted by the mathematical model. The targeted drug delivery system of the present invention has applications in the treatment of pathophysiological diseases or disorders for which radiation therapy is a useful treatment option. The present invention is particularly useful in delivering anti-vascularization or anti-angiogenic compounds to vascular endothelium.

The biomolecular carriers of the targeted delivery system include biodegradable particles, e.g., microspheres or microparticles and nanospheres or nanoparticles, liposomes, microbubbles, polymersomes and synthetic secretory granules. These biomolecular carriers may comprise molecules for stability such as, but not limited to, polyethylene glycol. Such molecules may be functionalized to link the targeting moiety to the biomolecular carrier. The biomolecular carriers are targeted to specific up-regulated ligands expressed on the luminal surface of irradiated endothelial cells.

Targeting moieties or molecules may be intact antibody, for example, a monoclonal antibody, or antibody fragments, such as Fab, Fv, F(ab′)₂, and sFv, that bind to cellular adhesion molecules. Targeting moieties also may comprise ligands that bind to cellular adhesion molecules. For example, a modified or cyclic RGD motif may be used to target upregulated integrins. Cellular adhesion molecules may include integrins, ICAM-1, E-selectin, P-selectin, VCAM-1 or PECAM-1. For example, a ligand may target an integrin β3 chain.

The biomolecular carrier encapsulates or otherwise carries a pharmaceutical to treat a pathophysiological state, such as a disease or disorder which, in itself, may be treated with radiation. The pharmaceutical may be used to treat a wide variety of pathophysiological states including, inter alia, cancer, arteriovenous malformations (AVM), macular degeneration and restenosis. Preferably, in the case of cancer, the pharmaceutical is an anti-neoplastic compound. More preferably, the pharmaceutical is an antiangiogenic compound, such as, but not limited to combretastatin and 5,6-dimethylxanthenone-4-acetic acid (DMXAA). Also it is contemplated that such pharmaceuticals may comprise suitable prodrugs. For other diseases or disorders commonly treated with radiotherapy any pharmaceutical effective against the disease or disorder may be used.

The biomolecular carriers of the present invention are administered to target irradiated tissue. The radiation may be administered as a single or fractionated dose. It is well within standard practice for one of ordinary skill in the art to determine the dose and dosage of radiation and of the pharmaceutical. Additionally, it is well within standard practice to determine an effective amount of pharmaceutical to be delivered by the biomolecular carriers. Such determinations are dependent upon, inter alia, the particular pathophysiological state and the degree of its advancement, the age and general health of the patient and any previous therapeutic treatment received.

The biomolecular carrier may be an immunoliposome, such as a stealth liposome comprising poly(ethylene)glycol copolymer (PEG). PEG is conjugated to the outer lipid bilayer comprising the liposome and is functionalized to link the targeting moiety to the liposome. Such linkers are known in the art, as for example, maleimide. Lipids used to form the liposome include a phosphatidycholine, cholesterol and a diacylphosphatidyl ethanolamine. For example, but not limited to such, hydrogenated soy phosphatidylcholine, cholestrol, and distearoylphosphoethanolamine may comprise a liposome.

Additionally, it is particularly contemplated that a biomolecular carrier comprising an immunoliposome be optimized for specific targeting to irradiated tissue. The immunoliposome may be designed such that the rate of binding to the irradiated tissue dominates the rate of uptake by the reticuloendothelial system (RES). Mathematical modeling provides a predictive tool to optimize a drug delivery approach. Additionally, these mathematical models may be used to elucidate the mechanisms governing the targeting of immunoliposomes to irradiated tumors.

Particular characteristics of the immunoliposomes may be modified according to the predictions of the mathematical model to further enhance the preferential delivery of pharmaceuticals, for example, but not limited to, antivascular drugs, to irradiated tumors by the immunoliposomes. A key point to consider is what effect liposome properties have on the rate of binding to target endothelium. For example, increasing the number of targeting ligands on the immunoliposomes may cause a significant increase in the rate at which the immunoliposomes will bind to irradiated tumor endothelium, while having very little effect on the rate at which the immunoliposomes are taken up by the RES. In this case a substantial increase would be achieved in the targeting of the immunoliposomes to the irradiated tumor. Other characteristics to consider are the effective diameter and polydispersity of the liposome. Mathematical modeling may be used to optimize other biomolecular carriers, such as, but not limited to, microspheres and nanospheres or other micro- or nano-particles.

It also is contemplated that the biomolecular carriers of the instant invention are useful for the targeted delivery of a therapeutic genetic macromolecule, such as, but not limited to, a gene, polynucleotide or other nucleic acid, to irradiated endothelial tissue. The therapeutic genetic macromolecule may comprise recombinant DNA. The therapeutic genetic macromolecule may comprise any suitable plasmid, vector or other construct known and standard in the art. It is well within the skill of one of ordinary skill in the art to use standard methods to construct a vector comprising a therapeutic gene, polynucleotide or other nucleic acid suitable for targeted delivery via a biomolecular carrier. The genetic macromolecule or construct comprising the genetic macromolecule expresses a therapeutic protein to treat a pathophysiological state for which radiation therapy is useful.

The following examples are given for the purpose of illustrating various embodiments of the invention and are not meant to limit the present invention in any fashion.

### EXAMPLE 1

General Animal Care

C57BLK mice, selected as a relatively inexpensive mammalian species to model targeted drug delivery to irradiated tissue, are utilized for in vivo experiments involving the cranial window preparation. Investigations concerning targeted drug delivery are conducted in an animal system since physiological changes and the resultant effects on the microvasculature are investigated in vivo to establish baseline data for the modeling studies.

Approximately 20 mice/month are purchased and housed for an average of 30 days. These mice are housed 2 per cage under 12 hr light/dark cycles with food and water ad libitum. Adult C57BLK mice are anesthetized with an i.m. injection of 87 mg of ketamine/kg and 13 mg of xylazine/kg. The body temperature is maintained between 36 and 37° C.

The cranial window is prepared for observation under an intravital microscope as discussed herein. The mice are euthanised by an overdose of KCl. Single or fractionated doses of 2-40 Gy of irradiation (Siemens MD-2 linear accelerator (6 mV X-rays) at the St. Jude Children's Hospital) are utilized to study targeted drug delivery to the irradiated tissue. Protocols typically involve observation of the microvasculature 1-30 days after single or fractionated doses of the ionizing radiation. The mice are anesthetized throughout the surgical procedure and data collection. Radiation is delivered locally to one hemisphere of the brain and the rest of the body is shielded.

### EXAMPLE 2

Statistics

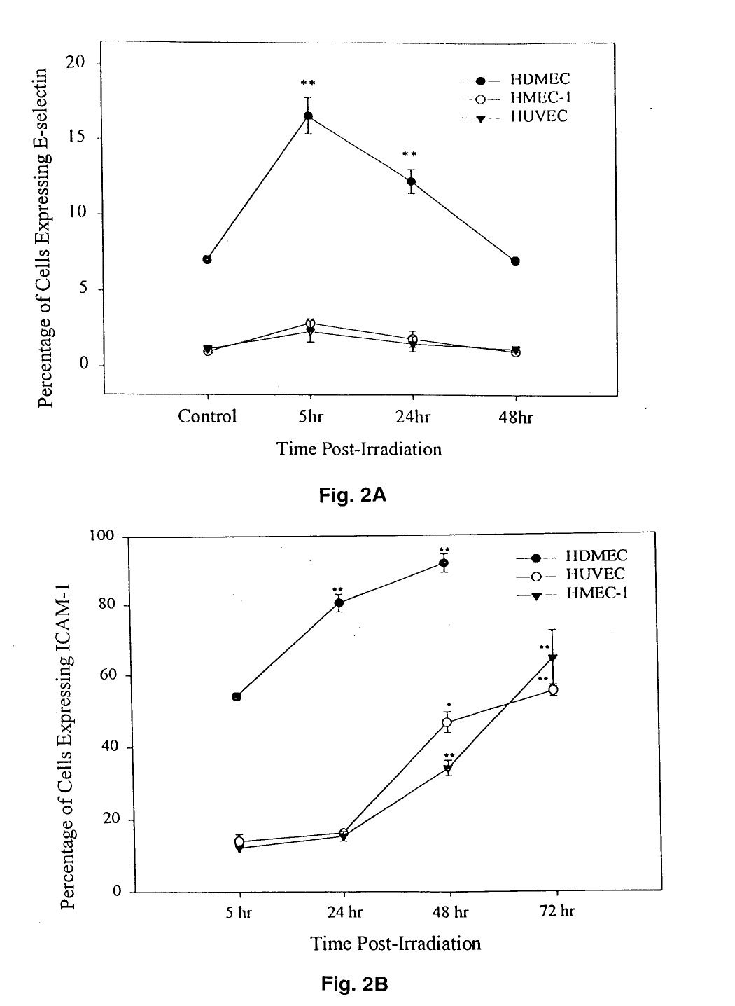

In FIGS. 2, 3, 5, 7, 8 and 11, significant difference from appropriate controls is indicated by * (p<0.05) or ** (P<0.01) as determined from one way analysis of variance (ANOVA) and a multiple comparison method (Fisher's least significant difference, LSD) to discriminate between the means. Data are presented as Mean±SEM.

### EXAMPLE 3

Generation of Ligand-Coated Polystyrene Particles

Due to their ease of use, polystyrene particles were used initially. The polystyrene particles were purchased from Bangs Laboratories (Fishers, Ind.). The particles are available in a variety of diameters, ranging from 20 nm to 10 mm, and with various incorporated fluorescent dyes. Since particles in the nanometer range cannot be detected by bright field light, fluorescent nanospheres were used and the fluorescent label was used to detect the nanospheres on a cellular surface.

The ligand coated polystyrene particles were prepared as follows. The particles were coated with protein A via passive adsorption. The particles were incubated in a 0.1 M NaHCO3, pH 9.2 buffer containing 300 mg/ml protein A at room temperature for over an hour. Following the adsorption, the particles are washed, incubated in a blocking buffer, i.e., Hank's balanced saline solution supplemented with 1% human or rat serum albumin, washed and incubated with a specific monoclonal antibody to an endothelial cell adhesion molecule also diluted in blocking buffer. After a 1 hour incubation, the monoclonal antibody coated particles are washed and stored in the blocking buffer prior to use in an assay.

Particles coated with a monoclonal antibody to ICAM-1 (R&D Systems; Minneapolis, Minn.) are generated initially. The surface density of monoclonal antibody on the particles is controlled by altering the amount of monoclonal antibody used in the coating step. The surface density of monoclonal antibodies on the particles is quantified via radiolabeling assays as described (69).

When working with microspheres, the washing steps, i.e., separation of the particles from solutions, are achieved via centrifugation. The concentration of microspheres in a solution is determined via a hemocytometer. When working with nanospheres, the separations are achieved via gel filtration. The concentration of nanospheres in a solution is determined via absorbance readings and comparison to a standard curve as described (6). These methods are well established (25) and generate ligand-coated particles.

### EXAMPLE 4

Anti-ICAM-1 Monoclonal Antibody-Coated Biodegradable Particles

Although the polystyrene particles are well suited for some studies, polystyrene is not a very relevant drug delivery carrier. A more physiologically relevant drug delivery carrier may comprise a variety of materials including biodegradable polymers. Recently much attention has been given to the use of particles made from biodegradable polymers as attractive drug carriers (5,16,28). Routine particles made from biodegradable polymers have two drawbacks. First they are rapidly removed from the circulation and second they apparently adsorb a low level of ligand (16). Dr. Shakesheff (University of Nottingham, Nottingham, UK) has generated a biotinylated PEGylated co-polymer that circumvents these problems.

Avidin chemistry is used to couple mAbs to particles made from this polymer (9). Microspheres from the biotinylated PEGylated co-polymer are utilized as these particles are easy to detect with bright field microscopy. Monoclonal antibodies are coupled to the co-polymer microspheres by first coupling avidin to the biotin covalently linked to the polymer. The microspheres are washed and incubated in a solution containing a biotinylated monoclonal antibody to an endothelial cell adhesion molecule, e.g. anti-ICAM-1. Again the microspheres are washed and then held in blocking buffer until used in an assay. The success of the coupling procedure is shown via adhesion assays as described herein. Ligand coated biodegradable nanospheres also are generated. Due to their small size, it is impossible to visualize the nanospheres with bright field microscopy. Thus a fluorescent tag is incorporated into the nanoparticles to allow their detection using fluorescently labeled avidin during the coupling procedure.

### EXAMPLE 5

Adhesion of anti-ICAM-1 Monoclonal Antibody Coated Particles to Cognate Adhesion Molecules In Vitro

After coupling the mAbs to the microspheres, in vitro adhesion assays determine if the particles exhibit selective adhesion to cognate presenting cellular monolayers. Static adhesion assays can test for a large number of conditions in a single assay. This simply determines if the ligand is coupled to the particles in such a way that the ligand is able to support adhesion to its cognate receptor. Systematic detailed studies are conducted of the interaction of the particles with the cognate receptor presenting surfaces under in vitro fluid dynamic conditions that mimic, in part, conditions present in vivo.

Adhesive substrates are prepared in 96 well plates. The adhesive substrates consist of human umbilical vein endothelial cells (HUVEC) treated with IL-1β 4 hours prior to the adhesion assays to elicit expression of inducible adhesion molecules, e.g. ICAM-1 on activated HUVEC, unactivated HUVEC as a negative control for activated HUVEC, tissue culture plastic coated with a recombinant purified form of a particular endothelial cell adhesion molecule, e.g. ICAM-1, or tissue culture plastic coated with blocking proteins, e.g. human serum albumin, as a negative control for tissue culture plastic coated with adhesion molecules.

The ligand-coated particles are added to the wells of the 96 well plates. After a set time of incubation, the wells are washed and the number of particles remaining in each well determined. For the microspheres the number of particles present under bright field microscopy is counted. For the nanospheres various fields of view under fluorescent illumination are recorded and then analyzing the intensity via image analysis. Particles coated with a control protein, such as human serum albumin, are added to certain wells instead of the ligand coated particles. In other wells, the adhesive substrates are pre-treated with fluid phase monoclonal antibodies prior to the introduction of the particles.

Testing the adhesion of the particles under the above listed conditions determines whether the ligand coated particles exhibit specific selective adhesion to substrates expressing the cognate endothelial cell adhesion molecule. For example, anti-ICAM-1 coated particles should show high levels of binding to activated HUVEC relative to unactivated HUVEC. This adhesion should be inhibited by pre-treatment of the HUVEC monolayers with fluid phase anti-ICAM-1, but not by pre-treatment with monoclonal antibody W6/32 which recognizes Class I. In addition, anti-ICAM-1 coated particles should bind to tissue culture plastic coated with ICAM-1 to a much greater extent than they bind to tissue culture plastic coated with human serum albumin.

Analysis of variance (ANOVA) tests for statistical significance of any observed differences. A multiple comparison procedure (Fisher's least significant difference, LSD) is used to discriminate among the means. Differences are considered statistically significant if P<0.05.

### EXAMPLE 6

Endothelial Cell Culture

Microspheres and nanospheres bearing mAbs to ICAM-1 are used since ICAM-1 is up-regulated by ionizing radiation. Other adhesion molecules, e.g. E-selectin, may also be used.

Human umbilical vein endothelial cells (HUVEC) and human dermal microvascular endothelial cells (HDMEC) are purchased from Colonetics, Inc. HUVEC are maintained in M199 supplemented with FBS, L-glutamine, heparin, endothelial growth factor and penicillin/streptomycin on gelatin coated tissue culture plastic. Confluent cells are trypsinized and subcultured at a ratio of 1:3. All studies are conducted on passage 3-6 of these cells.

HDMEC are maintained in MCDB131 media supplemented with human serum, FBS, L-glutamine, cyclic AMP, hydrocortisone acetate and penicillin/streptomycin. The cells are grown on 0.2% gelatin coated tissue culture dishes. Confluent cells are trypsinized and subcultured at a ratio of 1:3. All studies are conducted on passage 3-5 of these cells. For the assays described below, the endothelial cells are grown in 35 mm² tissue culture dishes.

### EXAMPLE 7

Irradiation and Flow Cytometry

Prior to irradiation, confluent endothelial cells are replenished with fresh media. HDMEC media are replaced with media lacking cyclic AMP which has been found to suppress the expression of E-selectin (26). Cells are irradiated with single doses of 10, 5, or 2 Gy or fractionated doses of 20-30Gy at 3Gy per day ionizing radiation at a dose rate of 4.2 Gy per minute. Cells taken to the radiation facility, but not irradiated, are used as controls. IL-1β (10 U/ml) activated cells serve as positive controls. Up-regulation of adhesion molecules on irradiated endothelial cells is probed using flow cytometry. Adhesion assays are conducted with cells 5 hr, 24 hr, 48 hr and 72 hr post irradiation.

Presence of various adhesion molecules on irradiated endothelial cells is probed via flow cytometry. At the respective time points 5 hr, 24 hr, 48 hr and 72 hr post-IR, endothelial cells are harvested from the tissue culture dish with 0.01% EDTA-trypsin mixture in the presence of 1% BSA, washed in phosphate buffer saline and incubated for 30 minutes at 4° C. with appropriate mAbs to endothelial cell adhesion molecules, e.g. monoclonal antibody to ICAM-1. Following the incubation, the endothelial cells are washed and incubated with secondary antibody, goat F(ab′)₂, FITC conjugated anti-mouse IgG, heavy and light Chain specific, for 30 minutes at 4° C. Subsequently, the cells are washed, are fixed in 2% formaldehyde and are analyzed by flow cytometry. Harvested endothelial cells treated with isotype non-specific murine IgG and not treated with a primary mAb serve as negative controls. Endothelial cells pre-treated with IL-1β 4 hr prior to the assays serve as positive controls.

### EXAMPLE 8

Quantify the Extent of Selective and Specific Adhesion/Deposition of the Carriers on Irradiated Endothelial Cells

A parallel plate flow chamber is used to study the interaction of the ligand coated particles with various adhesive substrates (14). The flow chamber has an inlet for the entry of the drug carriers, which are suspended in endothelial cell growth media, and an outlet connected to a syringe pump to control the flow rate. A second outlet is connected to a vacuum pump to seal the flow chamber and the 35 mm tissue culture dish containing the adhesive substrate. The height and width of the flow chamber, the viscosity of the media and the volumetric flow rate determine the wall shear stress in the flow chamber.

Once the flow chamber is sealed, it is transferred to the stage of an inverted phase contrast microscope. The microscope has an attached video camera connected to a VCR and monitor. Once on the microscope stage, the adhesive substrate is rinsed and the flow of the particle suspension initiated. The particles are coated with either a ligand for an endothelial cell adhesion molecule or non-specific IgG as a negative control. After a period of time, the images of several fields of view are recorded for later off-line analysis.

When microspheres are used as carriers, the images are taken under bright field light microscopy and the number of microspheres present is determined by simply counting the number of microspheres observed. When nanospheres are used as carriers, the images are recorded under fluorescent illumination. The images are imported into an image analysis workstation and the fluorescent intensity of the fields of view is determined to gain a semi-quantitative measure of the number of nanospheres present on the adhesive substrate.

The adhesive substrate consists of human umbilical vein endothelial cells, HUVEC, and human dermal microvascular endothelial cells, HDMEC. In certain assays these endothelial cells are treated with radiation prior to the adhesion assay. Negative control monolayers are prepared in exactly the same manner, but are not treated with radiation. Positive control monolayers are treated with IL-1β 4 hours prior to the adhesion assay to elicit expression of cytokine inducible endothelial cell adhesion molecules. In certain assays the endothelial cells are treated with fluid phase monoclonal antibodies prior to the introduction of the particles.

Several parameters are varied to gain insight into their effects on the selective adhesion. These parameters include the shear, the particle size, the ligand density and the ligand used to target a given endothelial cell adhesion molecule and the endothelial cell adhesion molecule targeted. Since the nanospheres may be endocytosed by the endothelial cells, certain assays are conducted at reduced temperature of 4° C. to gain insight into the role of endocytosis in the accumulation of the particles on/within the endothelial cells.

Testing the adhesion of the particles under the above listed conditions determines whether the ligand-coated particles exhibit specific-selective adhesion to post-irradiation endothelial cells relative to non-irradiated treated endothelial cells. For example, if anti-ICAM-1 coated biodegradable nanospheres exhibit much greater levels of binding to post-IR human umbilical vein endothelial cells relative to non-irradiated human umbilical vein endothelial cells and this augmented adhesion is inhibited by pre-treatment of the post-irradiated human umbilical vein endothelial cells with fluid phase anti-ICAM-1, but not pre-treatment with mAb W6/32 which recognizes MHC Class I, the data would indicate that the anti-ICAM-1 biodegradable nanospheres exhibit specific-selective adhesion to post-irradiated human umbilical vein endothelial cells.

By determining the ratio of binding to post-IR human umbilical vein endothelial cells relative to non-IR human umbilical vein endothelial cells, insight into the selectivity of the adhesion can be gained. By systematically altering the biophysical parameters, e.g. ligand density, shear, or targeting ligand, and determining the resulting effect on selectivity insight into what role these various parameters can have on the selective adhesion is gained. Note that the deposition of the particles on the surface of the endothelial cells is a function of several interrelated processes, e.g. transport and adhesion. Thus, to rationally interpret this data, one can use theoretical models that relate observed adhesion/deposition to adhesive mechanics (11,38) and transport (48).

### EXAMPLE 9

Targeted Delivery in a Mouse Closed Cranial Window Model

The mouse cranial window model is used herein as a model of normal tissue. The brain is a clinically relevant tissue in radiotherapy and pial vessels can be studied in an animal survival model using intravital microscopy techniques. Either left or right hemisphere of mice are locally irradiated at 7-8 weeks of age with the unirradiated hemisphere used as control. As an additional control, the interaction of the carriers with endothelium in each microvessel is measured before and after irradiation.

Prior to surgery animals are anesthetized with an i.m. injection of 15 μL of Ketaset (87 mg ketamine/mL+13 mg Xylazine/mL). Body temperature is maintained at approximately 37° C. by convective heating. Animals are placed on a small animal stereotaxic frame. All surgical procedures are carried out under aseptic conditions. The animal is prepped with three applications of iodine to the shaved scalp before the initial incision is made.

The scalp and tissue from a 1.5×1.5 cm area bilaterally over the parietal cortex is removed. Bleeding from soft tissue is controlled via heat cauterization, as needed, and the underlying fascia is blunt dissected. A circular cranial window extending from the coronal to the lambdoid sutures centered on the sagittal suture is traced using a dental drill at low speed. Care is taken to avoid frictional heat created by drilling for extended periods in any one area. Once the window has been sufficiently drilled out, the flap of bone is gently removed with forceps and the underlying tissue washed with repeated applications of sterile artificial cerebrospinal fluid (ACSF). Slight bleeding from bridging vessels is allowed to clot without intervention.

From this point on, all manipulations to the brain are carried out under a layer of sterile artificial cerebrospinal fluid. The dura is punctured with a 30 gauge needle and the tissue excised with microdissecting scissors as great care is taken not to make contact with the underlying brain tissue. Superficial bleeding is allowed to stop without intervention and the tissue is irrigated regularly with sterile artificial cerebrospinal fluid. A quartz plate resting on the bone surrounding the cranial window is glued to the surrounding bone using cyanoacrylate glue. After recovery from anesthesia windowed animals are returned to the animal facilities and are given one week to recover from surgery.

### EXAMPLE 10

Animal Irradiation

C57-black mice at 7-8 weeks of age weighing around 25 g are irradiated. Prior to irradiation animals are sedated with an i.m. injection of a mixture of 87 mg/kg ketamine and 13 mg/kg xylazine. A local single dose of 5, 10 or 20 Gy or fractionated doses of 20-40 Gy in 2 Gy daily fractions of radiation are delivered to randomly chosen left or right hemispheres of the brain at a rate of 2 Gy/min using a Siemens MD-2 linear accelerator (6 mV X-rays). A collimator 1.0 cm in diameter, which is normally used for human stereotactic radiosurgery, is used to localize the radiation dose to the left or the right brain. Tissue equivalent bolus is placed above and below the head to establish electronic equilibrium and to insure the prescribed dose is delivered uniformly to the brain.

### EXAMPLE 11

Intravital Microscopy Data Collection

Intravital microscopy techniques are used to compare the interaction of model fluorescent drug carriers and biodegradable drug carriers with endothelial cells in postcapillary venules in the irradiated brain hemisphere of each animal with the unirradiated or control hemisphere of the same animal (n=6-7 mice per group). These postcapillary venules are generally the site of up-regulation of adhesion molecules in response to irradiation and are usually in the range of 15-50 μm in diameter. The drug carriers are injected via tail vain.

All experiments are performed on a Nikon Measurescope MM-11 intravital microscope. Venule diameters are observed and recorded under reflected light illumination using a custom-designed epi-illumination filter cube with cross polarized excitation and emission filters including a band pass 550±20 nm excitation filter with a 100 W mercury lamp. Images are observed with a CCD camera in conjunction with an intensifier. Experiments are recorded on SVHS tape and analyzed offline using a computerized video imaging system.

### EXAMPLE 12

Control Experiments

A series of control experiments are performed to ascertain the preferential adhesion of antibody-coated microspheres to irradiated tissue. In one group of animals (n=6) the brain is irradiated locally with a 20 Gy local dose and adhesion of microspheres to the microvasculature of non-irradiated tissue, such as the cremaster, mesentery, liver, and lung, is compared to that of the brain using well-established intravital microscopy techniques (29,57,66). In another group of animals (n=6) the cremaster muscle, i.e., the testicular area, is irradiated locally with a 20 Gy local dose and adhesion of microspheres to the microvasculature of non-irradiated tissue, such as the brain, mesentery, liver, and lung, is compared to that of the cremaster. In these experiments antibody-bearing particles are injected directly into arteries upstream of the irradiated tissue, e.g., carotid artery for the brain and iliac artery for the cremaster.

Nanospheres bearing antibodies to ICAM-1 are used. E-selectin and other adhesion molecules also may be used. Initially, 2 μm red and blue fluorescent polystyrene microspheres, as model carriers, which bear ligands to adhesion molecules expressed on irradiated endothelial cells or control human IgG are used. The number of these microspheres interacting with the microvascular endothelium of irradiated tissue can be quantified easily by using dual filter fluorescent microscopy. By switching between red and blue fluorescent filter cubes the number of microspheres that bear ligands to adhesion molecules expressed on irradiated endothelial cells vs. control can be quantified. After verifying the enhanced interaction of fluorescent microspheres that bear ligands to adhesion molecules on their surface with irradiated tissue, biodegradable drug carriers are then used to selectively target irradiated endothelial cells. Fluorescent optical techniques as described above are used to quantify enhanced interaction of these drug carriers with irradiated tissue microvasculature.

### EXAMPLE 13

Up-Regulation of Adhesion Molecules on Irradiated Endothelial Cells In Vitro

The expression of E-selectin and ICAM-1 on human umbilical vein endothelial cells (HUVEC), human dermal microvascular endothelial cells (HDMEC) and transformed microvascular endothelial cells (HMEC-1) was investigated at 5 hr, 24 hr, 48 hr, and 72 hr post-irradiation. Both E-selectin and ICAM-1 have been implicated in the leukocyte adhesion cascade. E-selectin supports the attachment and rolling of leukocytes on the endothelium while ICAM-1 is involved in the firm adhesion of the leukocyte to the endothelium.

Flow cytometric analysis revealed significant up-regulation of E-selectin on human microvascular endothelial cells 5 and 24 hr. post-irradiation, using a 5-10 Gy single dose, but no up-regulation of E-selectin on human umbilical vein endothelial cells and HMEC-1 up to 48 hr post-irradiation (FIG. 2A). Consistent with these findings, in vitro flow assays revealed an increase in the rolling and adhesion of a leukocytic cell line HL60 on post-irradiation human microvascular endothelial cells, but no rolling of HL-60 cells on human umbilical vein endothelial cells and HMEC-1 monolayers post-irradiation. The increased rolling on post-irradiation human microvascular endothelial cells was reduced by more than 90% by pretreatment of the post-irradiation human microvascular endothelial cells with a mAb to E-selectin prior to introduction of the HL-60 cells (data not shown). Thus, it appears that E-selectin expression is up-regulated post-irradiation in some, but not all, in vitro endothelial cell models. Note that the literature is divided on the expression of E-selectin post-irradiation with one group reporting an increase (31-33) and others (21) reporting no expression of E-selectin post-irradiation.

In contrast to the variable results with E-selectin, ICAM-1 was up-regulated significantly in response to a single radiation dose of 5-10Gy on all three endothelial cell types tested (FIG. 2B). These results are consistent with a variety of reports (35-36,53,58-59) showing up-regulation of ICAM-1 in response to irradiation. Thus, the response of ICAM-1 to irradiation appears to be “robust”, i.e. occurring at several time points post-irradiation, occurring on all of the endothelial cells tested to date and being consistently reported as inducible post-IR.

### EXAMPLE 14

Up-Regulation of Leukocyte-Endothelium Interaction in Irradiated Tissue In Vivo

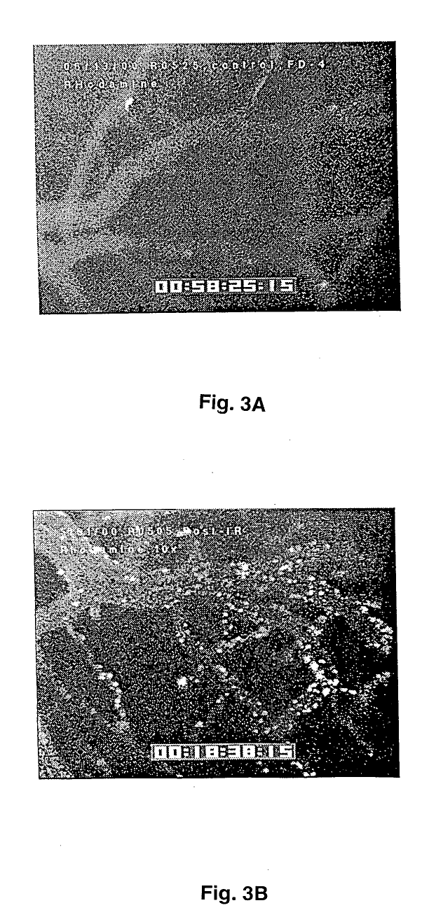

A closed cranial window model was used to determine the effects of a single 10Gy local dose of radiation on leukocyte-endothelial interactions in cerebral microvasculature in vivo. FIGS. 3A-3B show digitized pictures of rhodamine labeled leukocytes in the non-irradiated (FIG. 3A) and 48 hours post-irradiated (FIG. 3B) cerebral microvasculature in the closed cranial window model. The results (n=6 animals) indicate that the number of adhering leukocytes was elevated (˜124 leukocytes/mm²) at 2 hours post-irradiation and remained elevated up to 48 hours post-irradiation relative to control which stayed constant at ˜16 leukocytes/mm² (FIG. 3C) over the 2 hour to 48 hour time period studied. These results indicate that the up-regulation of leukocyte-endothelium interaction post-irradiation is present in vivo.

### EXAMPLE 15

Making and Characterizing Ligand Coated Particles

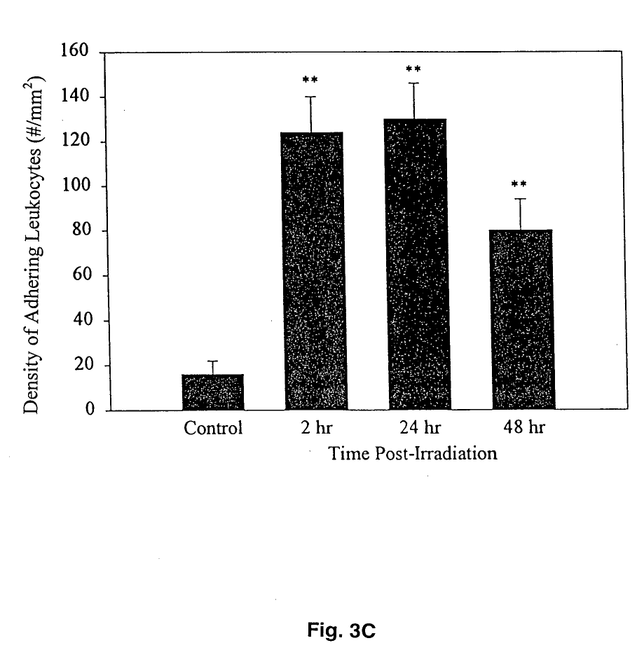

Significant research has focused on the development of ligand coated particles for use in adhesion assays (6,13,16,25,69). FIGS. 4A-4D demonstrate a typical result wherein 60 nm fluorescent (red) nanospheres were coated with either the monoclonal antibody HuEP5C7.g2 (HuEP) to E-selectin (40) or human IgG, as negative control, and allowed to adhere to Chinese hamster ovary cells stably expressing E-selectin (CHO-E). Bright field microscopy in FIGS. 4A and 4C show the CHO-E monolayers.

Fluorescent microscopy in FIGS. 4B and 4D reveal that the nanospheres coated with HuEP exhibit significantly higher levels of adhesion than nanospheres coated with human IgG; compare FIG. 4B to FIG. 4D. The nanospheres only are bound to the surface where CHO-E cells are present; compare FIG. 4A with FIG. 4B. This study was conducted with polystyrene particles which, although demonstrably relevant to binding studies, is not a very relevant drug delivery carrier. A more physiologically relevant drug delivery carrier could be made of a variety of materials including biodegradable polymers.

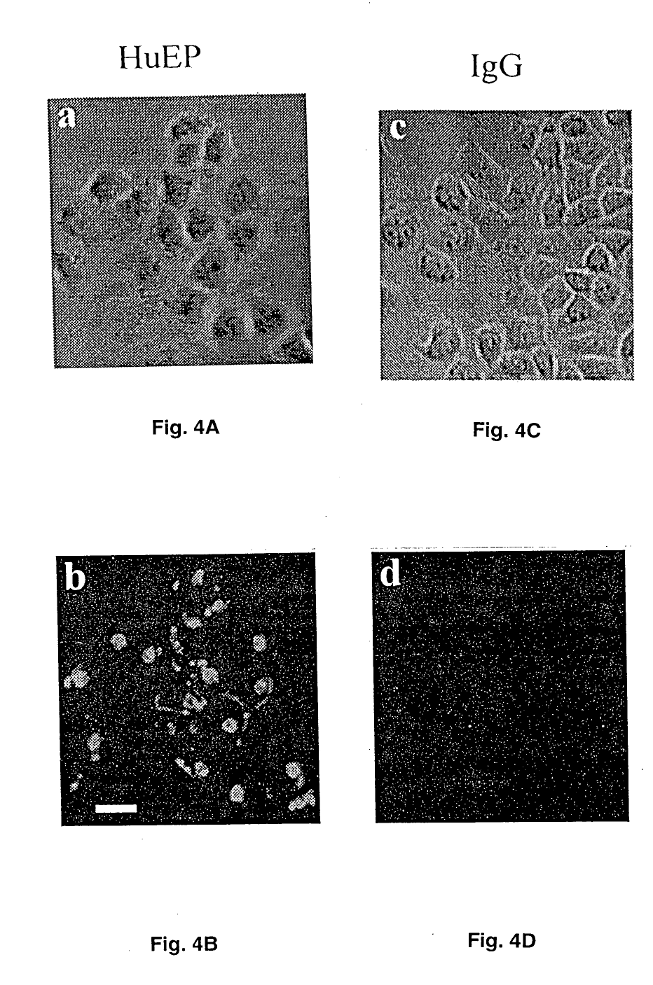

Consequently, monoclonal antibody HuEP5C7.g2 was passively adsorbed onto particles made from the biodegradable polymer poly-(e-caperlactone) (PCL) (16) and the adhesion of the resulting HuEP5C7.g2 PCL microspheres was studied. The HuEP5C7.g2 poly-(e-caperlactone) microspheres exhibit selective adhesion to activated HUVEC (A-HUVEC) relative to unactivated HUVEC (U-HUVEC) (FIG. 5) while poly-(e-caperlactone) microspheres coated with human IgG do not. The adhesion of the HuEP5C7.g2 poly-(e-caperlactone) microspheres was inhibited by pre-treatment of the A-HUVEC with a monoclonal antibody to E-selectin (HEL3/2), but unaffected by pre-treatment with endothelial cell binding mAb W6/32.

Although the adhesion appeared to be specific, the rate of attachment was quite low, occurring only under low shear, i.e., 0.3 dynes/cm², and at a rate estimated to be <1% of that exhibited by neutrophils. The low rate of attachment may be due to a low level of HuEP5C7.g2 coupled to the poly-(ε-caperlactone) microspheres via passive adsorption. Thus, particles made from a block copolymer of biotinylated poly(ethylene glycol) (PEG) with poly(lactic acid) (PLA) (9) can be used. Monoclonal antibody can be coupled to the particles via avidin-biotin chemistry allowing achievement of a high surface density of monoclonal antibody on the biodegradable particles (9). Additionally, particles made with poly(ethylene glycol) should have an enhanced circulation time.

### EXAMPLE 16

Enhanced Adhesion of Antibody Bearing Microspheres to Irradiated Endothelial Cells In Vitro

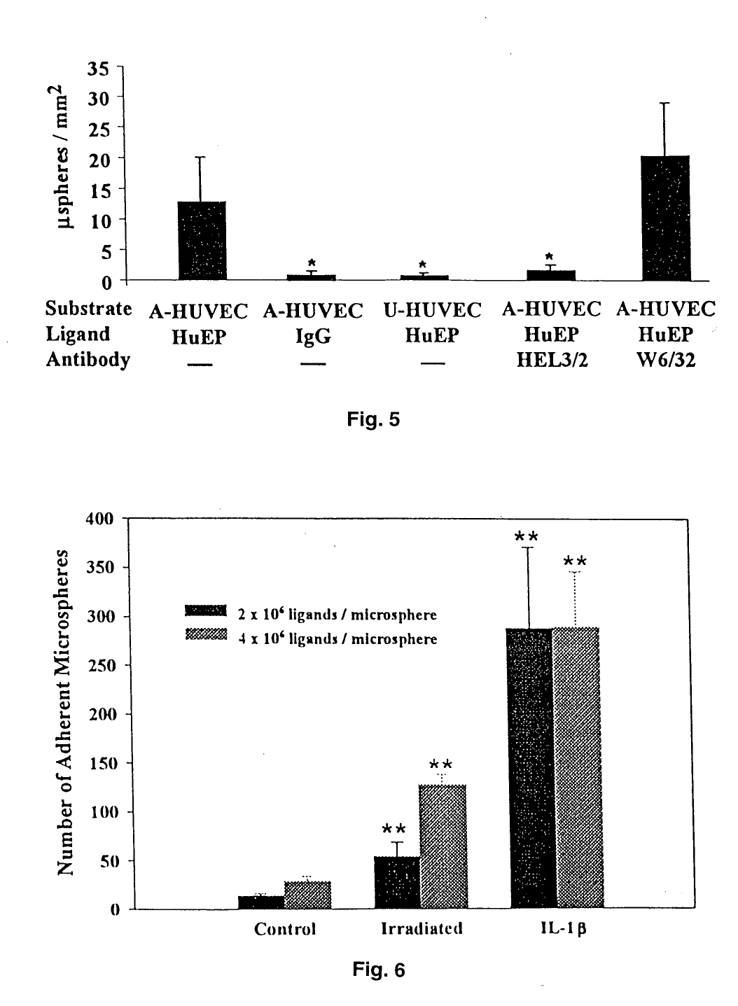

The interaction of antibody bearing polystyrene microspheres with irradiated endothelial cells was studied under static and shear flow conditions. The results indicate that under static conditions the number of adherent anti-ICAM-1 microspheres on 48 hr post-irradiated HUVEC was 4.9±1.8 (Mean±SEM) times that of control (P<0.01, N=3). Under shear flow conditions of 1.5 dynes/cm² the number of adherent anti-ICAM-1 microspheres on irradiated HUVEC was 3.9±1.2 to 4.5±0.9 times (P<0.01, N=3 in each group) that of control HUVEC, depending on the surface density of anti-ICAM-1 (FIG. 6). The selectivity of this targeting mechanism may be further enhanced by optimizing particle size, antibody density, etc.

### EXAMPLE 17

Enhanced Adhesion of ICAM-1 Antibody Bearing Micro Spheres to Irradiated Tissue In Vivo

The adhesion of polystyrene microspheres coated with a monoclonal antibody to ICAM-1 to cerebral microvasculature irradiated with 10Gy single local dose of X-ray was investigated in a rat closed cranial window model (n=4 animals). Fluorescent 2 mm diameter microspheres coated with either rat anti-ICAM-1 antibody or IgG, as negative control, were injected via tail vein into rats bearing closed cranial windows. Dual color fluorescent microscopy was used to quantify the level of adhesion of anti-ICAM-1 and IgG bearing microspheres to the cerebral venules before and after radiation.

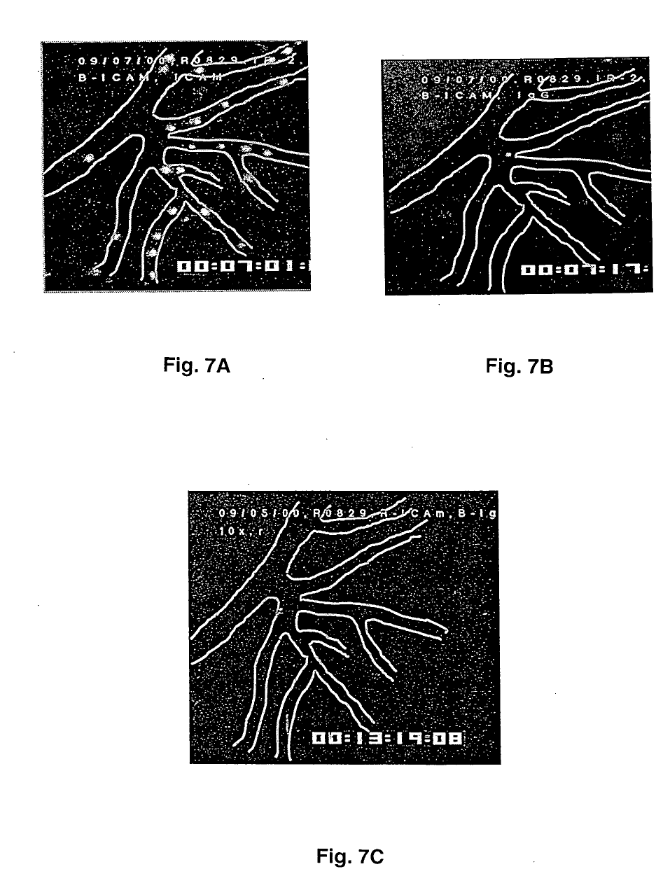

In the irradiated tissue a large number of anti-ICAM-1 coated microspheres adhere to the vessel wall (FIG. 7A), while very few IgG coated microspheres adhere to the walls of the same vessel (FIG. 7B). Also, there was very little adhesion of anti-ICAM-1 coated microspheres to the same vessels before this area of the brain was irradiated (FIG. 7C). Microvascular outline as determined from reflected light microscopy has been digitally superimposed on FIGS. 7A-7C.

The compiled data from the 4 animals revealed that the adhesion of anti-ICAM-1 coated microspheres to the irradiated cerebral microvasculature is up to 25 times higher than control and reaches a peak 48 hours post-irradiation (FIG. 7D). The number of adhering antibody bearing microspheres to sham irradiated microvasculature did not significantly differ from control up to 7 days post-irradiation (data not shown). The enhanced adhesion of antibody bearing microspheres to the irradiated tissue in vivo (FIG. 7D) is much more pronounced compared to the adhesion of antibody bearing microspheres in vitro (FIG. 6). The presence of red cells in vivo, which have been shown to enhance the interaction of particles with the endothelium (52,54), is the reason for this higher rate of adhesion. This can be shown in vitro with a flow chamber system using microspheres suspended in media containing red blood cells.

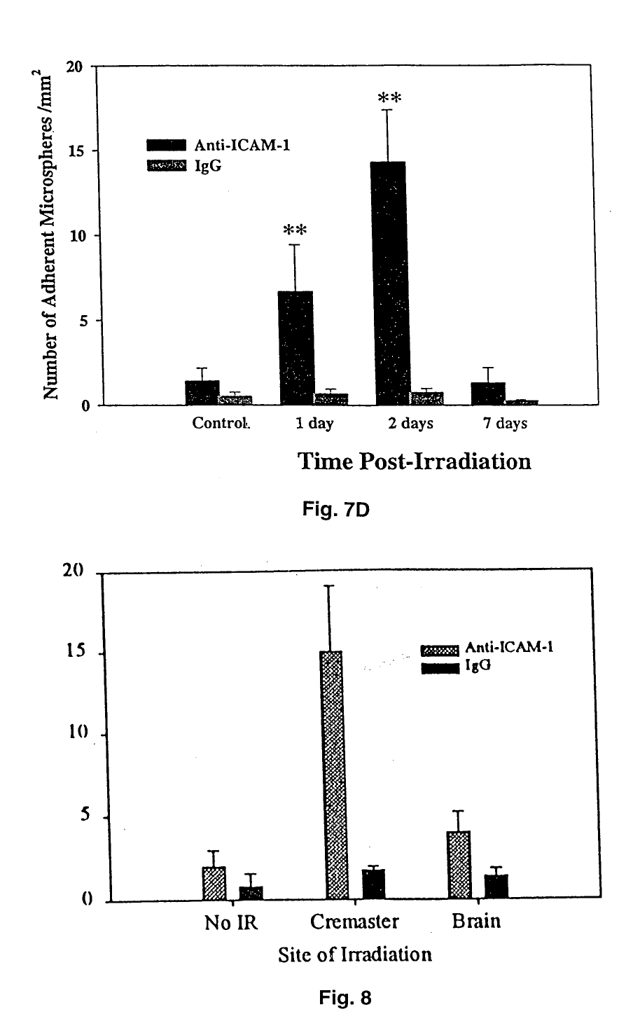

In a series of control experiments (n=2) the adhesion of the anti-ICAM-1 and IgG bearing microspheres to the microvasculature of the cremaster muscle in animals which received local irradiation only to the brain was investigated to ascertain the preferential adhesion of anti-ICAM-1 coated microspheres to irradiated tissue (FIG. 8). The results indicate that while the ratio of adherent anti-ICAM-1 coated microspheres was up to 25 times higher than that of IgG coated microspheres in the irradiated brain microvasculature, this ratio was only 2-3 times higher in the cremaster microvasculature. A basal level of anti-ICAM-1 coated microsphere adhesion to un-irradiated tissue is expected since a low level of ICAM-1 is constitutively expressed in all tissue under control conditions (27). The differential between the number of adherent particles to the irradiated brain microvasculature vs. the unirradiated cremaster microvasculature would presumably be increased by directly injecting the drug carrying particles to arteries upstream of the irradiated tissue, e.g., the carotid artery for the brain.

The possibility exists that leukocytes may compete with the endothelium for binding to the drug carriers because ICAM-1 exists not only on endothelial cells but also on leukocytes (10). To investigate this possibility leukocytes were labeled in vivo with rhodamine-6G (fluorescent in red) and their interaction with anti-ICAM-1 coated microspheres (fluorescent in blue) was observed using dual fluorescent microscopy. By rapidly switching between red and blue fluorescent filters, one could then determine if any leukocyte-microsphere doublets were either circulating or attached to the vessel walls. In two experiments, no adhesive interactions between anti-ICAM-1 coated microspheres and leukocytes in vivo, i.e., no doublets, were observed.

### EXAMPLE 18

Enhanced Adhesion of Selectin Antibody Bearing Microspheres to Irradiated Tissue In Vivo

The adhesion of polystyrene microspheres coated with mAb to E-selectin to irradiated (10Gy single local dose of X-ray) cerebral microvasculature in a rat closed cranial window model (n=5 animals/time point). Fluorescent 2 μm diameter microspheres coated with either rat anti-E-selectin antibody or IgG (negative control) were injected via tail vein into rat bearing closed cranial windows. Dual color fluorescent microscopy was used to quantify the level of adhesion of anti-E-selectin and IgG bearing microspheres to the cerebral venules before and after radiation.

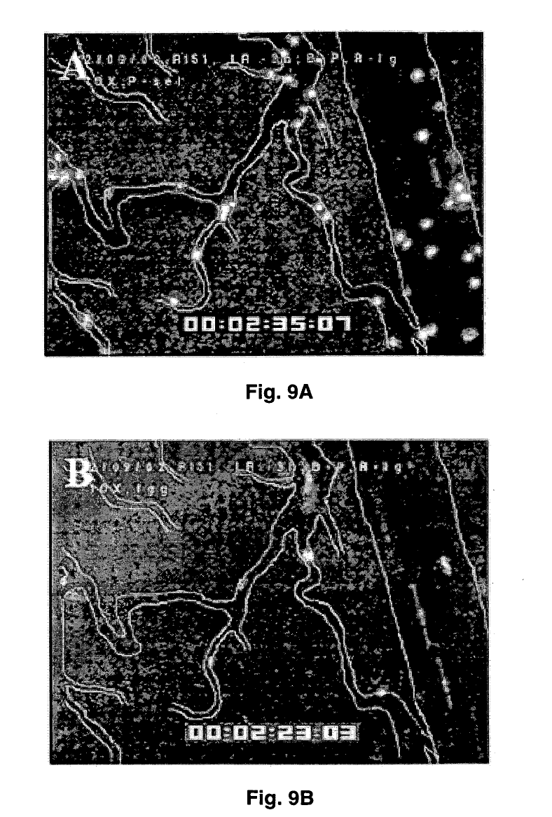

FIGS. 9A-9B show that in the irradiated tissue a large number of anti-E-selectin coated microspheres adhere to the vessel wall (FIG. 9A), while very few IgG coated microspheres adhere to the walls of the same vessel (FIG. 9B). The microvascular outline as determined from reflected light microscopy has been digitally superimposed on FIGS. 9A-9B. Also, very little adhesion of anti-E-selectin coated microspheres to the same vessels before this area of the brain was irradiated was apparent (data not shown).

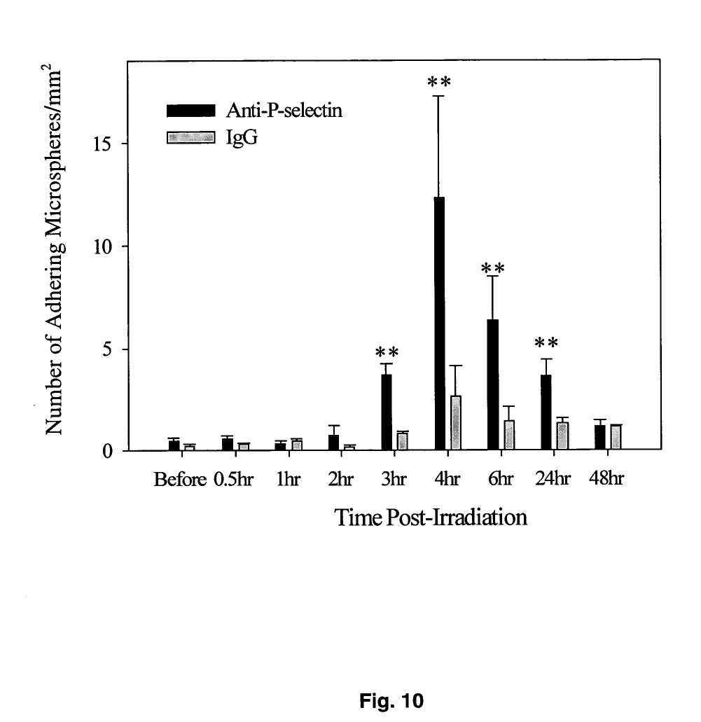

The compiled data from these animals revealed that the adhesion of anti-E-selectin coated microspheres to the irradiated cerebral microvasculature is 10 times, with a maximum of 25 times, higher than control and reaches a peak at 3 hours post-irradiation (FIG. 9C). The number of adhering antibody bearing microspheres to sham irradiated microvasculature did not significantly differ from control up to 48 hours post-irradiation (data not shown). Radiation also upregulated the adhesion of model drug carriers bearing rat anti-P-selectin to irradiated tissue, but the peak was at 4 hours post-IR (FIG. 10). It is contemplated that preferential delivery of antivascular drugs to normal tissue in the tumor margin would potentiate the effect of the drug in the tumor itself.

### EXAMPLE 19

Targeting PEGylated Microspheres to Tumors and Their Lack of Adhesion to Liver

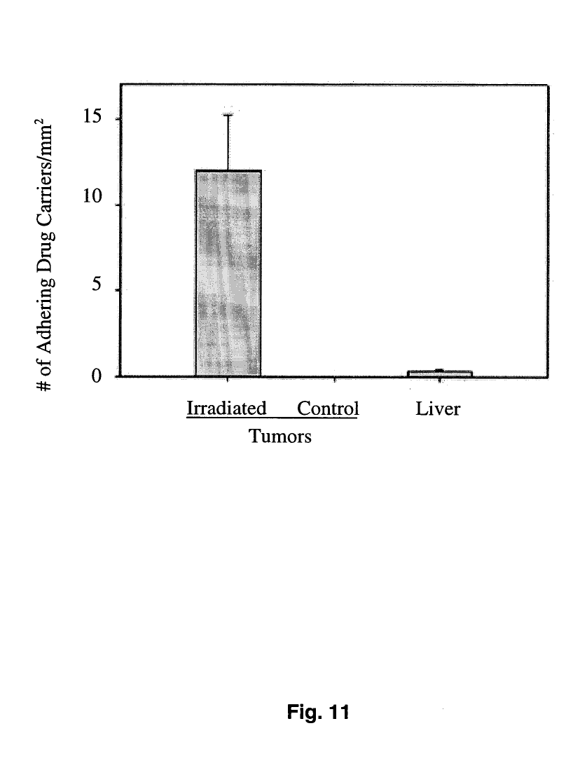

PEGylated microspheres (2 μm diameter) bearing ligands to E-selectin were injected into animals (N=2) having an irradiated (6 hrs post-IR) B16-F10 melanoma tumor in one leg and a non-irradiated tumor in the other leg. The level of adhesion of PEGylated microspheres to both tumors and the liver was measured. As shown in FIG. 11, while a large number of these microspheres attached to the irradiated tumor, there was almost no adhesion to irradiated tissue vs. control. These data strongly suggest that PEGylated drug carriers can be selectively targeted to irradiated tissue while sparing un-irradiated, i.e., normal, tissue.

### EXAMPLE 20

Composition and Characterization of Immunoliposomes

Stealth liposomes comprised hydrogenated soy phosphatidyl choline (HSPC), cholesterol, and distearoyl phosphoethanolamine-PEG(2000) conjugate (DSPE-PEG). The liposome surface was covered with PEG chains to prolong their blood circulation time. For preparation of stealth immunoliposomes, part of the PEG lipid was replaced with maleimide functionalized PEG-lipid. Liposomes were made by the standard lipid film hydration and extrusion process. Thiol containing cyclic RGD peptide was coupled covalently to the distal end of PEG chains through the maleimide functional group. An antiangiogenic, antivascular drug, combretastatin A4, was incorporated into the liposome bilayer. Combretastatin A4 as a prodrug for combretastatin comprises phosphate groups for added water solubility.

Immunoliposomes were analyzed for size and charge uniformity on a Brookhaven 90Plus Submicron Particle Size Analyzer with a Zeta Potential Analyzer. The results indicate that the immunoliposomes have an effective diameter of 122.5 nm with a polydisperity of 0.051 with an average zeta potential of −48.67 mv. The zeta potential of immunoliposomes did not change significantly after many weeks of storage, but their polydisperity started to increase after one week of storage at 4° C., possibly due to the aggregation of the proteins on the surface on the immunoliposomes. Therefore, the immunoliposomes are used only during the first week after their production.

### EXAMPLE 21

Targeting Antivascular Drugs to Irradiated Tumors

B16-F10 melanoma tumors, 0.8-1.1 mm³ volume, in hind leg of C57BL mice (n=4 per group) were treated with various combinations of low dose (5Gy) radiotherapy plus antivascular drugs: systemic i.v. combretastatin (final concentration of 3.2 mg/mouse) therapy (Sys), radiation (IR), Sys+IR, combretastatin (final concentration of 0.32 mg/mouse) encapsulated in RGD deficient liposomes (Lipo), Lipo+IR, combretastatin (final concentration of 0.32 mg/mouse) encapsulated in immunoliposomes carrying a cyclic RGD sequence (Arg-Gly-Asp-D-Phe-Cys (SEQ ID NO: 1; custom made by Peptides International) on their surface targeting the β₃ chain on irradiated tumor endothelial cells (IL), and IL+IR.

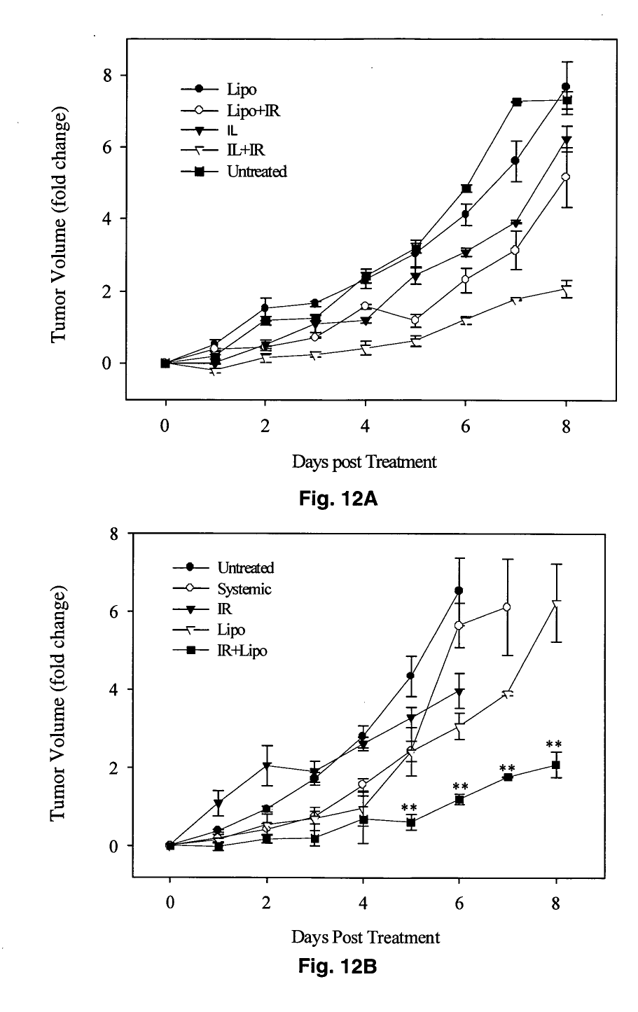

There was no significant difference between the tumor growth curves of untreated tumors and Sys groups. Also, no significant differences between IR and Lipo+IR groups was found (data not shown). However, as shown in FIG. 12A, while IR caused a slight growth delay in tumors, IL+IR caused a significant growth delay. Treating irradiated tumors via immunoliposomes resulted in a significant growth delay while treatment with a 10 times larger systemic dose did not result in a tumor growth delay.

Larger size tumors (1.2-1.5 mm³) which may be less vascularized and more radio-resistant (79). Our results indicate that targeted delivery of combretastatin (12.8 mg/kg) to irradiated (low dose of 5Gy) B16-F10 melanoma tumors (IR+Lipo) (n=5-6 per group) caused a significant tumor growth delay (see FIG. 12B). Other treatment combinations including radiation alone (IR), systemic treatment (Sys, 128 mg/kg), or liposome alone (Lipo, 12.8 mg/kg) regiments did not cause a significant growth delay in these tumors. This demonstrates efficacy over a wide range of tumor sizes is an indication of the versatility of this unique combined radiation/antivascular therapy.

### EXAMPLE 22

Targeting Antivascular Drugs to Spontaneous Tumors

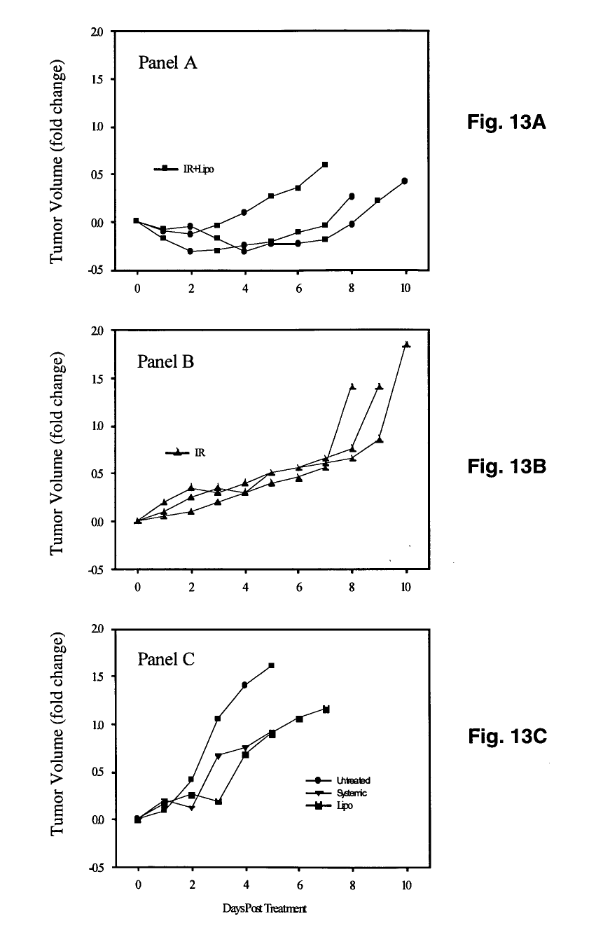

Animals (n=1-3) bearing spontaneous mammary tumors were treated with targeted delivery of combretastatin encapsulated in liposomes having RGD on their surface targeting the P3 chain on irradiated tumor endothelial cells to irradiated tumors. These tumors have been treated with either targeted delivery of combretastatin to irradiated tumors (IR+Lipo at 12.8 mg/kg), radiation alone (IR), systemic combretastatin alone (Sys at 128 mg/kg), immunoliposome containing combretastatin alone (IL at 12.8 mg/kg), or no treatment (FIG. 13A). This data clearly indicate that while irradiation caused a slight growth delay in these tumors (FIGS. 13A & 13C), targeted delivery of antivascular drugs to irradiated spontaneous tumors caused a much larger significant growth delay in these tumors (FIGS. 13A & 13B) compared to transplanted tumors. Systemic treatment or liposome alone regiments did not cause a significant growth delay in these tumors (FIGS. 13A & 13C).

These significant tumor growth delays are achieved with only a single IR/immunoliposome treatment cycle. It is contemplated that these growth delays can be significantly enhanced with repeated IR/immunoliposome treatment cycles. Thus, radiation targeting of combretastatin can significantly enhance its tumoricidal effects while at the same time reducing its systemic toxicity. Also, it is contemplated that compared to the B16-F10 transplanted tumors, these mammary spontaneous tumors are much more slow growing and better vascularized and, therefore, are more amenable to targeting aimed at endothelial cell adhesion molecules.

### EXAMPLE 23

Vascular Perfusion in Treated and Untreated Tumors

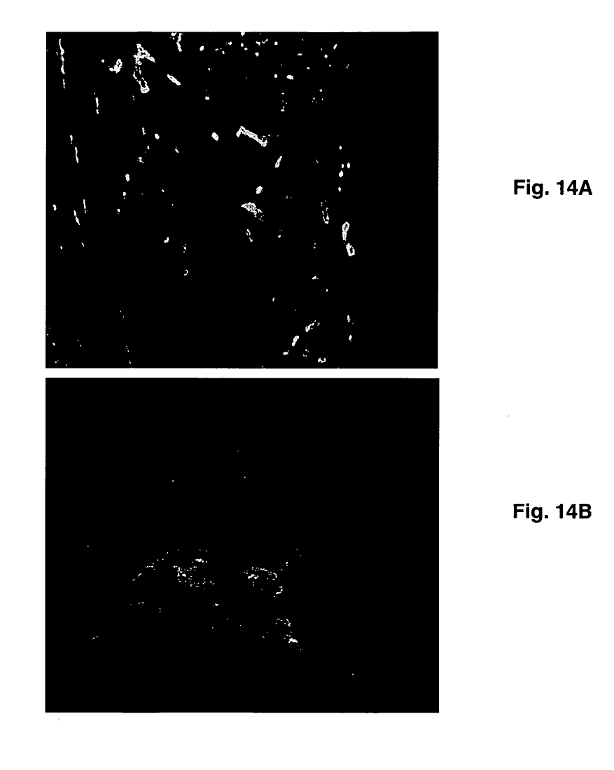

The fluorescent carbocyanine dye, 3,3′-diheptyloxacarbocyanine (DiOC7) (Molecular Probes, Eugene, Oreg.) was administered in untreated and IR+IL treated animals 24 hours post IR to fluorescently label perfused vessel. A significant reduction in the number of perfused vessels in treated (FIG. 14B) vs. untreated tumors (FIG. 14A) is observed. Compared to untreated tumors there is a 66% reduction in the number of perfused vessels (107/field vs. 36/field) and a 72% increase in distance to the nearest perfused vessel (49 μm vs. 177 μm) in treated tumors (n=2 animals per group).

In comparing the B16-F10 transplanted tumors, the vasculature in mammary transplanted tumors are better perfused and are less necrotic. The number of perfused vessels=61/mm² for spontaneous tumors s. 1/mm² for B16-F10 transplanted).

### EXAMPLE 24

Normal Tissue Toxicity of Targeted Antivascular Drugs

In the previous review cycle of this grant application one of the reviewers suggested that “normal adjacent muscle should be studied for toxicity”. We have taken this suggestion as an opportunity to not only study the normal tissue toxicity of the proposed targeted drug delivery system but also to determine the mechanism by which targeted delivery of antivascular drugs to irradiated tumors may enhance tumor shrinkage and growth delay.

Selected organs, i.e., brain, heart, kidneys, liver, lung, spleen, pancreas, intestine, tumors, as well as skin and normal muscle adjacent to the tumor of treated by targeted delivery of combretastatin to irradiated tumors (IR+Lipo) and untreated spontaneous tumors (n=2) were harvested and examined by a certified animal pathologist (Dr. Stanley D. Kosanke, Oklahoma University Health Science Center Animal Resources) for evidence of normal tissue toxicity. While a very large growth delay was observed in treated tumors (FIGS. 13A-13B & 13D), no evidence of normal tissue toxicity in the treated animals was observed, not even in the normal tissue adjacent to the treated tumors. Targeting antivascular drugs to irradiated tumors is not only efficacious in treating tumors, but also can circumvent the undesirable side effects of these drugs in many normal tissue even those in the vicinity of tumors being treated.

A significantly greater amount of necrosis was observed in the treated tumors as compared to the untreated tumors. This is consistent with the vascular perfusion observed (FIGS. 14A-14B) and the vascular shutdown mechanism of action of combretastatin with subsequent necrosis. It is contemplated that the combination of the tumor vascularity and perfusion measurement techniques with the detailed pathological examinations provide a powerful mechanistic tool for studying the mechanisms of this targeted drug delivery system.

### EXAMPLE 25

A Mathematical Model for Targeted Drug Delivery to Irradiated Tumors

Targeting a drug to irradiated endothelium and its effects on tumor growth can be significantly enhanced if drug carrier characteristics are optimized. A mathematical model describing targeted immunoliposome delivery to irradiated endothelium can be used for optimizing immunoliposomal characteristics. The parameters that can affect binding to the targeted irradiated endothelium are included in a single parameter that describes the rate at which particles bind to the target endothelium. Similarly, the parameters that can affect non-specific uptake, e.g. by the RES, are represented by a first order rate constant. A parameter, τ, is defined as the ratio of the inclusive adhesion rate constant (K_{ad}) to the target endothelium relative to the rate constant (Kᵣₑₛ) for uptake of particles by the RES. Thus,
τ=Kₐₛ/Kᵣₑₛ.

Calculations based on this

$$τ = \frac{{K}_{ad} / {K}_{res}}{{k}_{res}} .$$

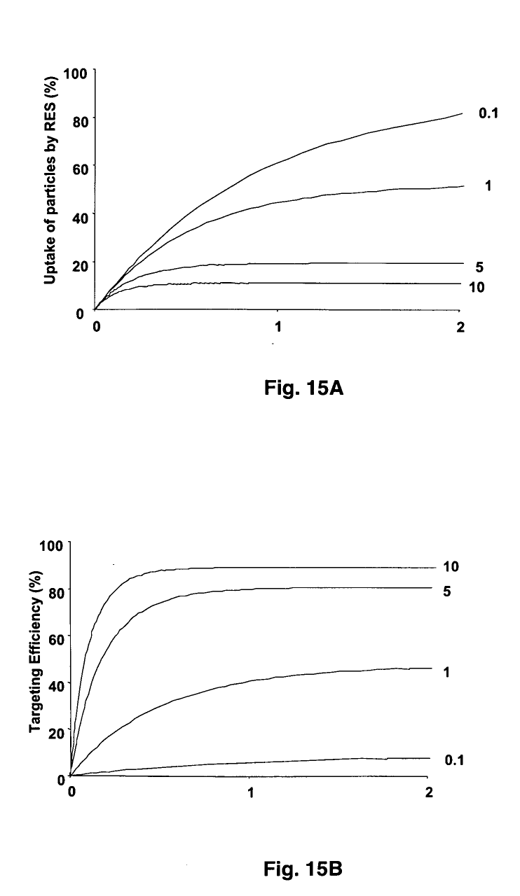

simplified model dramatically illustrate how altering the relative rates can significantly affect the targeting. For example, the percentage of particles that are taken up by the RES (FIG. 15A) or that bind to the target endothelium (FIG. 15B) each can be plotted as a function of dimensionless time for various values of τ=0.1, 1, 5, 10. As shown, if Kᵣₑₛ dominates, e.g. τ=0.1, the percentage of particles that are targeted to the irradiated endothelium is less than 10%, while greater than 80% go to the RES. The total doesn't add up to 100% because steady state has not been reached at this time point. However, if Kᵣₑₛ is less dominant, e.g. τ=1, then nearly 50% of the particles are targeted. If binding to the endothelium dominates, e.g. τ=5, then >80% of the particles are targeted to the target endothelium while less than 2% go to the RES.

This model clearly indicates that τ has a significant effect on selective delivery. Importantly, τ can be increased by decreasing the rate of uptake by the RES or by increasing the rate of binding to the target endothelium. Decreasing uptake by the RES has been the subject of numerous studies (80-81). Similar techniques, e.g. modifying pegylation levels, can be used to increase the circulation time of immunoliposomes, taking into consideration that pegylation level and liposome membrane integrity are coupled.

The following references were cited herein:
1. Acker et al., Radiat. Res. 149: 350-359, 1998.
2. Behrends et al., J. Invest. Dermatol. 103: 726-730, 1994.
3. Bendas et al., Int J Pharm 181: 79-93, 1999.
4. Bendas et al., Pharm Acta Helv 73: 19-26, 1998.
5. Benoit et al., Int J Pharm 184: 73-84, 1999.
6. Blackwell et al., Ann Biomed Eng. June; 29(6):523-33, 2001.
7. Bloemen et al., FEBS Letters 357: 140-144, 1995.
8. Buell et al., Dig. Dis. Sci. 34: 390-399, 1989.
9. Cannizzaro et al., Biotechnol Bioeng 58: 529-535, 1998.
10. Carlos et al., Blood 84: 2068-2101, 1994.
11. Chang et al., Biophys. J. 76: 1280-1292, 1999.
12. Chiang et al., Int. J. Radiat. Biol. 72: 45-53, 1997.
13. Crutchfield et al., J. Leuk. Biol. 67: 196-205, 2000.
14. Crutchfield et al., J. Leukoc. Biol. 67: 196-205, 2000.
15. Diamond et al., Current Biology 4: 506-517, 1994.
16. Dickerson et al., Biotechnol Bioeng. 73(6): 500-9, Jun. 20, 2001.
17. Discher et al. Science 284: 1143-1146, 1999.
18. Ebnet et al. Histochem. Cell Biol. 112: 1-23, 1999.
19. Eldor et al. Prostaglandins Leukotrienes Essential Fatty Acids 36: 251-258, 1989.
20. Fajardo et al., Pathol. Annu. 23: 297-230, 1988.
21. Gaugler et al., Int. J. Radiat. Biol. 72: 201-209, 1997.
22. Gobbe et al., Radiat. Res. 130: 236-240, 1992.
23. Goetz et al., Am. J. Pathol. 149: 1661-1673, 1996.
24. Goetz et al., Int J Cancer 65: 192-199, 1996.
25. Goetz, et al., J. Cell Biol. 137: 509-519, 1997.
26. Goldsmith et al., Thrombosis and Haemostasis 55(3): 415-435, 1986.
27. Grange, et al., Physiology and Pathophysiology of Leukocyte Adhesion. New York, Oxford University Press. 1995.
28. Gref et al., Science 263: 1600-1603, 1994.
29. Hahn et al., Cancer Res. 52: 1750-1753, 1992.
30. Hallahan et al., Oncology (Huntingt) 13: 71-77, 1999.
31. Hallahan et al., Biochem. Biophys. Res. Commun. 217: 784-795, 1995.
32. Hallahan et al., Cancer Res. 56: 5150-5155, 1996.
33. Hallahan et al., Radiat. Res. 147: 41-47, 1997.
34. Hallahan et al., Cancer Res. 58: 5216-5220, 1998.
35. Hallahan et al., Proc. Natl. Acad. Sci. USA 94: 6432-6437, 1997.
36. Hallahan et al., Cancer Res. 57: 2096-2099, 1997.
37. Hallahan et al., Radiat. Res. 152: 6-13, 1999.
38. Hammer et al., Biophys. J. 62: 35-57, 1992.
39. Handschel et al., Int. J. Radiat. Oncol. Biol. Phys. 45: 475-481, 1999.
40. He et al., J. Immunol. 160: 1029-1035, 1998.
41. Heckmann et al., Exp. Cell Res. 238: 148-154, 1998.
42. Hong et al., Int. J. Radiat. Oncol. Biol. Phys. 33: 619-626, 1995.
43. Kansas G., Blood 88: 3259-3287, 1996.
44. Kimura et al., Int. J. Radiat. Oncol. Biol. Phys. 33: 627-633, 1995.
45. Kiser et al., Nature 394: 459-62, 1998.
46. Kwock et al., Am. Rev. Respir. Dis. 125: 95-99, 1982.
47. Kyrkanides et al., J. Neuroimmunol. 95: 95-106, 1999.
48. Lok et al., J. Coll. Inter. Sci. 91: 104-116, 1983.
49. Luscinskas et al., Annu. Rev. Med. 47: 413-421, 1996.
50. Martin et al., Int. J. Radiat. Oncol. Biol. Phys. 10: 1903-1906, 1984.
51. Matzner et al., J. Immunology 140(8): 2681-2685, 1988.
52. Melder et al., Microvasc. Res. 59: 316-322, 2000.
53. Molla et al., Int. J. Radiat. Oncol. Biol. Phys. 45: 1011-1018, 1999.
54. Munn et al., Biophys. J. 71: 466-478, 1996.
55. Needham et al., Cancer Res. 60: 1197-1201, 2000.
56. Newton et al., J. Leukoc. Biol. 61: 422-426, 1997.
57. Nguyen et al., Radiat. Res. 2000.
58. Olschowka et al., Brain Behav. Immun. 11: 273-285, 1997.
59. Panes et al., Gastroenterology 108: 1761-1769, 1995.
60. Prabhakarpandian et al., Nanotech, 1:92-95, 2002.
61. Qin et al., Am. J. Clin. Oncol. 20: 263-265, 1997.
62. Qin et al., Int. J. Radiat. Oncol. Biol. Phys. 19: 1507-1510, 1990.
63. Quarmby S et al Arterioscler. Thromb. Vasc. Biol. 19: 588-597, 1999.
64. Riccardi et al., Clin. Cancer Res. 4: 69-73, 1998.
65. Rose et al., J. Surg. Oncol. 49: 231-238, 1992.
66. Roth N M and Kiani M F. Ann. Biomed. Eng. 27: 42-47, 1999.
67. Roth et al., Radiat. Res. 151: 270-277, 1999.
68. Russell et al., Cancer Treat. Rev. 25: 365-376, 1999.
69. Shinde et al., submitted 2000.
70. Slatkin et al., Med. Phys. 19: 1395-1400, 1992.
71. Spragg et al., Proc. Natl. Acad. Sci. 94: 8795-8800, 1997.
72. Springer T A, Cell 76: 301-314, 1994.
73. Van Der et al., Cytokine 11: 831-838, 1999.
74. Villanueva et al., Circ. 98: 1-5, 1998.
75. Witte et al., Cancer Res. 49: 5066-5072, 1989.
76. Wu et al., Brit. J. Cancer 69(5): 883-889, 1994.
77. Yuan et al., Brain Res., 969(1-2): 59-69, 2003.
78. Zimmermann et al., Strahlenther. Onkol. 174 Suppl 3: 62-65, 1998.
79. Landuyt et al., Int. J. Radiat. Oncol. Biol. Phys. 49 (2):443-450, 2001.
80. Oku N and Namba Y, Crit Rev Ther Drug Carrier Syst 11: 231-270, 1994.
81. Torchilin V P, Immunomethods 4: 244-258, 1994.
82. Sakhalkar et al., Proc. Natl. Acad. Sci. U.S.A., 100(26):15895-15900, 2003.
82. Patil et al., Biophys J., 80(4):1733-43, April 2001.

Any patents or publications mentioned in this specification are indicative of the levels of those skilled in the art to which the invention pertains. These patents and publications are herein incorporated by reference to the same extent as if each individual publication was specifically and individually by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

1. A targeted delivery system, comprising:
a biomolecular carrier;
a targeting moiety specific for cellular adhesion molecules expressed on endothelial cells conjugated to said biomolecular carrier; and
a pharmaceutical contained by said biomolecular carrier.

2. The targeted delivery system of claim 1, further comprising a functionalized poly(ethylene)glycol copolymer on said biomolecular carrier linked to the targeting moiety.

3. The targeted delivery system of claim 2, wherein said functional group is maleimide.

4. The targeted delivery system of claim 1, wherein said biomolecular carrier is a biodegradable particle, a liposome, a microbubble, a polymersome, or a synthetic secretory granule.

5. The targeted delivery system of claim 1, wherein said targeting moiety is an antibody or fragment thereof or ligands that bind to said cellular adhesion molecule.

6. The targeted delivery system of claim 5, wherein said ligand binds to an integrin β3 chain.

7. The targeted delivery system of claim 6, wherein said ligand is a cyclic Arg-Gly-Asp-D-Phe-Cys peptide.

8. The targeted delivery system of claim 1, wherein said cellular adhesion molecule is an integrin, ICAM-1, E-selectin, P-selectin, VCAM-1, or PECAM-1.

9. The targeted delivery system of claim 8, wherein said integrin is an integrin β3 chain.

10. The targeted delivery system of claim 1, wherein said pharmaceutical is an anti-neoplastic compound, an anti-angiogenic compound or a therapeutic genetic macromolecule.

11. The targeted delivery system of claim 10, wherein said anti-angiogenic compound is combretastatin or 5,6-dimethylxanthenone-4-acetic acid (DMXAA) or a prodrug thereof.

12. The targeted delivery system of claim 10, wherein said therapeutic genetic macromolecule is a gene.

13. The targeted delivery system of claim 1, wherein said biomolecular carrier is a liposome, said targeting moiety is a cyclic Arg-Gly-Asp-D-Phe-Cys peptide and said pharmaceutical is an anti-angiogenic compound.

14. The targeted delivery system of claim 13, wherein said anti-angiogenic compound is combretastatin or 5,6-dimethylxanthenone-4-acetic acid (DMXAA) or a prodrug thereof.

15. A method of selectively targeting endothelial tissue for delivery of a pharmaceutical to an individual, comprising:
irradiating a target tissue or organ in the individual;
expressing cellular adhesion molecules on a luminal surface of endothelial tissue comprising said irradiated target tissue or organ;
administering the targeted delivery system of claim 1 comprising the pharmaceutical to said individual; and
selectively binding the targeting moiety comprising said targeted delivery system to said cellular adhesion molecule thereby delivering the pharmaceutical to said individual.

16. The method of claim 15, further comprising:
treating a pathophysiological state of said target tissue or organ with said pharmaceutical.

17. The method of claim 16, wherein said pathophysiological state is a cancer, arteriovenous malformations, macular degeneration or restenosis.

18. A method of treating a pathophysiological state in an individual in need of such treatment, comprising:
irradiating a target tissue or organ characterized by said pathophysiological state in said individual;
administering the targeted delivery system of claim 1 to said individual; and
delivering the pharmaceutical comprising said targeted delivery system to said irradiated target tissue or organ thereby treating the pathophysiological state in the individual.

19. The method of claim 18, wherein said pathophysiological state is a cancer, arteriovenous malformations, macular degeneration or restenosis.

20. A targeted delivery system, comprising:
a liposome;
a cyclic Arg-Gly-Asp-D-Phe-Cys peptide conjugated to said liposome; and
a pharmaceutical encapsulated by said liposome.

21. The targeted delivery system of claim 20, wherein said liposome comprises a phosphatidyl choline, cholesterol and a diacylphosphatidylethanoloamine-poly(ethylene)glycol conjugate.

22. The targeted delivery system of claim 21, wherein said phosphatidyl choline is hydrogenated soy phosphatidylcholine and said diacylphosphatidylethanoloamine-poly(ethylene)glycol conjugate is distearoylphosphoethanolamine-PEG(2000).

23. The targeted delivery system of claim 20, wherein said pharmaceutical is an anti-neoplastic compound or an anti-angiogenic compound.

24. The targeted delivery system of claim 23, wherein said anti-angiogenic compound is combretastatin or 5,6-dimethylxanthenone-4-acetic acid (DMXAA) or a prodrug thereof.

25. A method of optimizing an immunoliposome for specific targeting of a pharmaceutical encapsulated therein to irradiated tissue, comprising:
selecting at least one lipid or liposomal component and a targeting moiety such that the combination thereof forms a targeted liposome having a rate of adhesion (Kad) to said irradiated tissue greater than a rate of uptake (Kres) by the reticuloendothelial system wherein a majority of said targeted liposomes specifically adhere to the irradiated tissue thereby delivering the pharmaceutical thereto.

26. The method of claim 25, wherein a ratio (τ) of Kad/Kres is greater than 1.

27. The method of claim 26, wherein said ratio (τ) is greater than 5.

28. A targeted immunoliposome produced by the method of claim 25.
